(19) United States
(12) Patent Application Publication
(10) Pub. No.: US 2003/0157486 A1
(43) Pub. Date: Aug. 21, 2003

(54) Methods to identify signal sequences

(76) Inventors: Jonathan M. Graff, Dallas, TX (US); Matthew Muenster, Irving, TX (US)

Correspondence Address: BAKER BOTTS L.L.P., 30 ROCKEFELLER PLAZA, NEWYORK, NY, 10112, US

(21) Appl. No.: 10/002,631

(22) Filed: Oct. 31, 2001

Related U.S. Application Data
(60) Provisional application No. 60/300,309, filed on Jun. 21, 2001.

(51) Int. Cl. C12Q 1/68; C12N 1/21; C12N 15/74

(52) U.S. Cl. 435/6; 435/471; 435/252.3

(57) ABSTRACT

The present invention provides screening methods using bacterial cells to identify nucleic acid sequences encoding eukaryotic proteins comprising signal sequences and/or transmembrane sequences. Provided are several breast cancer and adipose tissue nucleic acid and proteins sequences that encode proteins comprising signal sequences and/or transmembrane sequences. Also provided are diagnostic methods and kits that utilize the proteins identified by the present methods to diagnose and detect diseases, physiological states and conditions, including cancer and those associated with fat metabolism.

### BACKGROUND OF THE INVENTION

The present application claims priority to co-pending U.S. patent application Ser. No. 60/300,309, filed Jun. 21, 2001. The entire text of each of the above-referenced disclosures is specifically incorporated by reference herein without disclaimer.

1. Field of the Invention

The present invention relates to the fields of identification of eukaryotic proteins comprising signal sequences and/or transmembrane domains. More particularly, it concerns the development of screening assays using prokaryotic cells to identify eukaryotic polypeptides that comprise signal sequences and/or transmembrane sequences and isolating and identifying their corresponding nucleic acid sequences.

2. Description of Related Art

Secreted proteins, extracellular proteins and transmembrane proteins have important functions such as transmitting and receiving information between cells as well as from the immediate environment. Transmission of information is accomplished by secreted polypeptides such as, hormones, growth factors, differentiation factors, cytotoxic factors, neuropeptides, and the like. Receipt and interpretation of information is most often accomplished by a variety of transmembrane proteins such as, various cellular receptors, ion channels, and other signal transducing proteins. Both, secreted polypeptides and transmembrane proteins normally pass through specialized cellular secretion pathways to reach their site of action in the extracellular or transmembrane regions.

The targeting of both secreted and transmembrane proteins to the specialized cellular secretory pathways is accomplished by the presence of a short, amino-terminal sequence, known as the signal peptide or signal sequence or leader sequence (von Heijne, 1985; Kaiser & Botstein, 1986). The signal peptide or signal sequence comprises elements necessary for protein targeting to an appropriate location. Although several proteins comprising signal sequences are known, there is no consensus DNA sequence that commonly identifies a signal sequence.

As signal sequence-containing proteins include the vast majority of signaling proteins and their receptors, they constitute an important group of proteins that are ideal for therapy or as targets for drug discovery. In addition, these proteins are also involved in cell adhesion, cell migration, and cell metastasis in cancer. Furthermore, identification of signal sequences allows the generation of secreted proteins by recombinant DNA methods. Obtaining secreted proteins is of importance in commercial protein production to obtain a variety of proteins including enzymes, hormones, drugs, etc. Yet another important utility of identifying proteins comprising signal sequences, is in the diagnosis of diseases. Most proteins that circulate in the blood stream comprise a signal protein or are secreted proteins and are therefore ideal targets for diagnostic blood tests.

Several methods to screen for signal sequences are described in the art. One of these methods described in European Patent Number EP0244042 to Smith et al. provides a system that utilizes Bacilli for detecting prokaryotic signal sequences involved with secretion in unicellular prokaryotic organisms.

Yet other methods describe yeast-based systems. For example, Klein R. D. et al., (1996), and U.S. Pat. No. 5,536,637, describe identification of cDNAs encoding novel secreted and membrane-bound mammalian proteins by detecting their secretory leader sequences using the yeast invertase gene as a reporter system. Accordingly, a mammalian cDNA library is ligated to a DNA encoding a yeast invertase gene that has been engineered to remove the secretory sequences, the ligated DNA is isolated and transformed into yeast cells that lack the invertase gene. Recombinants containing the nonsecreted yeast invertase gene ligated to a mammalian signal sequence are then identified based upon their ability to grow on a medium containing only sucrose or only raffinose as the carbon source. As invertase catalyzes the breakdown of sucrose and raffinose, the secreted form of invertase is required for utilization of sucrose/raffinose. Thus, cDNAs comprising mammalian signal sequences are identified and a second round of screening the library allows the isolation of clones encoding the corresponding secreted proteins. However, the invertase yeast selection process has a major disadvantage in that there is need for a certain threshold level of invertase activity that is required to allow growth on sucrose or raffinose media. This threshold level is about 0.6-1% of wild-type invertase secretion and all mammalian signal sequences are not capable of functioning to yield this amount of invertase secretion (Kaiser, C. A. et al. (1987).

U.S. Pat. No. 6,060,249, describes another yeast-based screening method, where mammalian signal sequences are detected based upon their ability to effect the secretion of a starch degrading enzyme such as amylase, lacking a functional native signal sequence. The secretion of the enzyme is monitored by the ability of the transformed yeast cells, which cannot degrade starch naturally or have been rendered unable to do so, to degrade and assimilate soluble starch.

The major deficiencies of the yeast-based systems of screening is the requirement of two-step procedures for screening. Additionally, yeast cells are complicated organisms to manipulate and their growth rates are slow. This makes the screening procedures time consuming, technically demanding, and expensive.

Proteins that comprise a transmembrane sequence and/or a signal sequence (i.e., proteins that are either secreted from the cell or reside on the surface of the cell), are ideal targets for blood tests for the diagnosis of diseases. For example, blood levels of the prostrate specific antigen (PSA), a cell-surface protein, is currently used to screen for prostate cancer. Therefore these molecules are useful for blood tests. But before such blood screening tests are developed, one must identify disease-specific or disease-related molecules that may be screened. Unfortunately, no technology currently exists to easily, generally, and quickly identify molecules that mark the onset of major diseases. As the discovery of novel secreted and transmembrane proteins provides potential diagnostic and therapeutic agents for a wide variety of diseases there is a great need for an improved system which can simply and efficiently identify the coding sequences of such proteins.

### SUMMARY OF THE INVENTION

The present invention overcomes these and other defects in the art and provides methods for identifying and isolating polypeptides and nucleic acids encoding polypeptides comprising a signal sequence and/or a transmembrane sequence using prokaryotic systems.

Therefore, provided are methods of screening candidate eukaryotic nucleic acid for one or more nucleic acid sequence encoding a signal sequence and/or a transmembrane sequence comprising: a) providing a bacterial cell; b) contacting the bacterial cell with at least one plasmid comprising a candidate eukaryotic nucleic acid segment and a marker gene comprising a mutation in a region comprising a signal sequence and/or a transmembrane sequence of the marker gene; and c) screening for function of the marker gene; wherein function of the marker gene indicates that the candidate nucleic acid segment comprises a sequence that encodes a signal sequence and/or a transmembrane sequence.

The term ‘signal sequence’ is defined herein as a sequence that targets or selects a peptide/polypeptide/protein to the cells secretory pathway. It will be appreciated by one of skill in the art that ‘polypeptides comprising a signal sequence’ are not necessarily always secreted proteins but also include those polypeptides that are targeted to the secretory machinery of the cell (i e., transmembrane or cell surface). Thus, the polypeptides that may be identified by the methods of the invention include polypeptides that may be either secreted, or targeted to the secretory machinery for processing or those that are membrane-bound polypeptides.

It is contemplated that the methods will be useful to identify a wide variety of eukaryotic nucleic acid molecules. Therefore, the candidate nucleic acid may be derived from any eukaryotic source.

In some embodiments of the methods, the nucleic acid is invertebrate nucleic acid. In specific non-limiting examples, the invertebrate nucleic acid is fly nucleic acid, or C. elegans nucleic acid.

In other embodiments, the nucleic acid is vertebrate nucleic acid. In other specific embodiments, the vertebrate nucleic acid is amphibian nucleic acid. Non-limiting examples of the amphibian nucleic acid is frog nucleic acid. Other examples of the vertebrate nucleic acid is reptilian nucleic acid, avian nucleic acid, or mammalian nucleic acid. Non-limiting examples of mammalian nucleic acid include mouse nucleic acid and human nucleic acid.

Additionally, the nucleic acid may be derived from any cell or tissue within a eukaryotic organism. Thus, in some specific, but non-limiting examples, the nucleic acid is fat cell nucleic acid, breast cell nucleic acid, blood cell nucleic acid, thyroid cell nucleic acid, pancreatic cell nucleic acid, ovarian cell nucleic acid, prostate cell nucleic acid, colon cell nucleic acid, bladder cell nucleic acid, lung cell nucleic acid, liver cell nucleic acid, stomach cell nucleic acid, testicular cell nucleic acid, uterine cell nucleic acid, brain cell nucleic acid, lymphatic cell nucleic acid, skin cell nucleic acid, bone cell nucleic acid, kidney cell nucleic acid, rectal cell nucleic acid, pituitary cell nucleic acid.

In some specific embodiments, the nucleic acid is a cancer cell nucleic acid and is derived from a cancer cell. In some embodiments, the cancer cell may be obtained from a tumor. In other embodiments, the cancer cell is from an immortal cancer cell line. In yet other embodiments, the cancer cell nucleic acid is breast cancer nucleic acid, hematological cancer nucleic acid, thyroid cancer nucleic acid, melanoma nucleic acid, T-cell cancer nucleic acid, B-cell cancer nucleic acid, ovarian cancer nucleic acid, pancreatic cancer nucleic acid, prostate cancer nucleic acid, colon cancer nucleic acid, bladder cancer nucleic acid, lung cancer nucleic acid, liver cancer nucleic acid, stomach cancer nucleic acid, testicular cancer nucleic acid, an uterine cancer nucleic acid, brain cancer nucleic acid, lymphatic cancer nucleic acid, skin cancer nucleic acid, bone cancer nucleic acid, kidney cancer nucleic acid, rectal cancer nucleic acid, sarcoma cancer nucleic acid, pituitary cancer nucleic acid, lipoma nucleic acid, adrenalcarcinoma nucleic acid, or nerve cell cancer nucleic acid.

In some embodiments of the invention, the breast cancer nucleic acid is breast cancer cell line nucleic acid, or an immortalized breast cancer cell line and may be exemplified by MCF7 nucleic acid, SKBR-3 nucleic acid, MDA-MB-231 nucleic acid, MCF6 nucleic acid, T47D nucleic acid, or MDA-MB-435 nucleic acid. In other embodiments, it is contemplated that the breast cancer nucleic acid is a breast cancer sample nucleic acid.

A ‘sample’ is defined herein as a cell, cellular extract, tissue, tissue extract, biopsy sample, a needle core biopsy, blood, lymph, plasma, urine, saliva, seminal fluid, or any biological fluid obtained from a subject that is a patient or suspected to have a disease, physiological condition or any other condition.

In other embodiments, the invention contemplates that the nucleic acid may be derived from a cultured cell.

In yet other embodiments, the nucleic acid is plant nucleic acid, such as one exemplified by corn, wheat, tobacco, arabidopsis, soybean, rice, or canola nucleic acid.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 2 and about 100 nucleobases in length. The term “polynucleotide” refers to at least one molecule of greater than about 100 nucleobases in length.

In some aspects of the invention, the marker gene is further defined as a selectable marker gene comprising a mutation in a region comprising a signal sequence and/or a transmembrane sequence of the marker gene, and screening for function of the marker gene is further defined as assaying for survival of the cell or its progeny cells on the selectable media. In some embodiments, the survival of the cell or its progeny on selectable media indicates that the candidate nucleic acid sequence encodes a polypeptide comprising a signal sequence and/or a transmembrane sequence.

In other embodiments, the methods of the invention further comprise isolating at least one nucleic acid segment comprising a nucleic acid sequence encoding a polypeptide comprising a signal sequence and/or a transmembrane sequence from the candidate nucleic acid. In some specific aspects, the methods are further defined as comprising isolating a plurality of nucleic acid segments comprising sequences encoding a polypeptide comprising a signal sequence and/or a transmembrane sequence from the candidate nucleic acid.

The methods may further comprise identifying at least one isolated nucleic acid segment. In some aspects, the identifying comprises sequencing the nucleic acid sequence. In other aspects, the identifying comprises expressing the nucleic acid sequence and identifying any polypeptides expressed. In some specific aspects, the polypeptides expressed can be identified using antibodies. Various different antibodies are contemplated including, polyclonal antibodies, monoclonal antibodies, conjugated antibodies, unconjugated antibodies, etc. In some embodiments, it is contemplated that the antibodies used for identifying will be prepared by phage display technology. Methods for making and using antibodies are well known to the skilled artisan.

The invention also envisions the use of cell-based assays for identifying. Such assays can comprise detecting the changes in cell sizes or shapes, induction of apoptosis, induction of chemotaxis, induction of cellular motility, induction of gene expression and activation of reporters. Additionally, biochemistry-based assays may be used for the identification such as phosphorylation, dephosphorylation and complex formation. One of ordinary skill in the art is well versed with such assays and methods.

In some embodiments, the methods further comprise characterization of at least one isolated nucleic acid segment. In some aspects, the methods comprise characterization of a plurality of isolated nucleic acid segments. The characterization of nucleic acids can be accomplished by various methods. For example, the characterization can comprise a microarray analysis, or Northern blot analysis, or reverse transcriptase-polymerase chain reaction (RT-PCR™). In other examples, the characterization comprises expression of a polypeptide encoded by at least one candidate nucleic acid segment. The polypeptide expressed can then be identified by various methods known to the skilled artisan. For example, function of the polypeptide can be analyzed or the antigenicity of the polypeptide may be determined.

In some aspects, the methods of the invention comprise determining whether the nucleic acid sequence or any polypeptide it encodes is an indicator of a disease, state of physiological condition, or other condition. The various diseases contemplated include hematological diseases, cardiovascular diseases, neurological diseases, renal diseases, hepatic diseases, gasterointestinal diseases, endocrinological diseases, oncological diseases, pulmonary, rheumatological diseases, etc. Non-limiting examples of such diseases include, cancers, Alzheimer's disease, osteoporosis, coronary artery disease, congestive heart failure, stroke, or diabetes. Many states of physiological conditions are also contemplated, for example, the state of fat metabolism. In some specific embodiments, the characterization is further defined as determining whether the nucleic acid sequence or any polypeptide it encodes is an indicator that a subject has a disease, state of physiological condition, or other condition. In other specific embodiments, the characterization is further defined as determining whether the nucleic acid sequence or any polypeptide it encodes is an indicator that a subject has a propensity for a disease, state of physiological condition, or other condition. In some aspects, the methods further comprise determining that the nucleic acid sequence or any polypeptide it encodes is an indicator of a disease, state of physiological condition, or other condition. In other aspects, the methods further comprise assaying a subject for the nucleic acid sequence or any polypeptide it encodes to determine whether the subject has or has a propensity for a disease, state of physiological condition, or other condition. In yet other aspects, the methods further comprise determining that the subject has or has a propensity for a disease, state of physiological condition, or other condition.

The bacterial cell that may be used is a gram negative or gram positive bacterial cell. Examples of such bacteria include Acetobacter, Acinetobacter, Bacillus, Brevibacterium, Campylobacter, Citrobacter, Clostridium, Corynebacterium, E. coli, Enterobacter, Heliobacter, Klebsiella, Lactobacillus, Leuconostoc, Micrococcus, Pseudomonas, Staphylococcus, Streptococcus, Thiobacillus or Vibrio. In specific embodiments, the bacteria is E. coli. In other specific embodiments, the bacteria is a Bacillus and is exemplified by B. subtilis, B. thuringiensis, B. stearothermophilus, B. licheniformis.

The invention contemplates the use of a wide variety of marker genes. In some embodiments, the marker gene can be a screenable marker gene, a scorable marker gene, a measurable marker gene, or a selectable marker gene. These marker genes may be detectable by fluorescence methods, colorimetric methods, or enzymatic methods. In some embodiments, the marker gene is a scorable marker gene and is exemplified in non-limiting examples by the chloramphenicolacetyl transferase gene, luciferase gene, or green fluorescent protein (GFP). In other embodiments, the marker gene is a screenable marker gene and is exemplified in non-limiting examples by a fluorescent protein gene, or a beta-galactosidase gene. In yet other embodiments, the marker gene is a selectable marker gene and is exemplified by but not limited to, an antibiotic resistance gene, a multidrug resistance gene, an herbicide resistance gene, or a toxin resistance gene. In still other embodiments, the selectable marker gene is an antibiotic resistance gene, for example, a beta-lactamase gene, or a multidrug resistance gene. In some preferred embodiments, the antibiotic resistance gene is a beta-lactamase gene and is, but not limited to, an ampicillin-resistance gene, a penicillin-resistance gene, a cephalosporin-resistance gene, an oxacephem-resistance gene, a carbapenem-resistance gene, or a monobactam-resistance gene. In specific embodiments where the beta-lactamase gene is an ampicillin-resistance gene the screening process may comprise growth selection on selective media.

In some aspects of the methods of the invention, the mutation in a region comprising a signal sequence and/or a transmembrane sequence of the marker gene, is a deletion in the signal sequence of the marker gene. In specific aspects, the mutation is a deletion of the entire signal sequence of the marker gene. In other aspects, the mutation is an insertion in the signal sequence of said marker gene. In yet other aspects, the mutation is a frameshift mutation in the signal sequence of said marker gene. In still other aspects, the mutation is a truncation of the signal sequence of said marker gene.

In some embodiments, the bacterial cell comprises a second marker gene such as, but not limited to, a kanamycin resistance gene.

In other embodiments, the candidate nucleic acid is DNA. The candidate DNA can be comprised in a DNA library. Various types of DNA libraries can be used as the candidate DNA and include genomic DNA libraries, oligonucleotide librararies, or cDNA libraries. In some aspects of the methods, at least two members of the library are screened. In other aspects, at least 10 members of the library are screened. In yet other aspects, at least 100 members of the library are screened. In still other aspects, at least 1000 members of the library are screened. In further aspects, at least 10,000 members of the library are screened. In another aspect, the entire library is screened.

It is also contemplated that a cloning site may be operably positioned in relation to the marker gene. Such a cloning site comprises at least one restriction site. Alternatively, the cloning site may comprise a multiple cloning site The multiple cloning site may comprise from 2 to 10,000 restriction sites. Thus, a multiple cloning site may comprises at least 2, 5, 10, 15, 20, 25, 30, 35, 40, 45, 50, 55, 60, 65, 70, 75, 80, 85, 90, 95, 100, 200, 300, 400, 500, 600, 700, 800, 900, 100, 2000, 3000, 4000, 5000, 6000, 7000, 8000, 9000, up to at least 10,000 restriction sites. Intermediate numbers of restriction sites are also contemplated, such as 3, 4, 101, 102, 1001, 1002, etc. In other aspects, the candidate nucleic acid is cloned into the plasmid by TA cloning.

The invention also provides methods of screening candidate nucleic acid for one or more nucleic acid sequence encoding a polypeptide comprising a signal sequence and/or a transmembrane sequence comprising: a) providing a bacterial cell; b) contacting the bacterial cell with at least one plasmid comprising a candidate nucleic acid segment and a marker gene comprising a mutation in a region comprising a signal sequence and/or a transmembrane sequence of the marker gene; and c) screening for function of the marker gene; wherein function of the marker gene indicates that the candidate nucleic acid segment comprises a sequence that encodes a polypeptide comprising a signal sequence and/or a transmembrane sequence.

Additionally, provided are methods of screening candidate nucleic acid for one or more nucleic acid sequences encoding a polypeptide comprising a signal sequence and/or a transmembrane sequence comprising: a) providing a bacterial cell; b) contacting the bacterial cell with at least one construct comprising a candidate nucleic acid segment and a mutated selectable marker gene comprising a mutation in a region comprising a signal sequence and/or a transmembrane sequence of the marker gene; and c) screening for survival of the cell on selectable media; wherein survival of the cell or its progeny cells on the selectable media indicates that the candidate nucleic acid segment comprises a sequence encoding a polypeptide comprising a signal sequence and/or a transmembrane sequence.

The invention also provides constructs for screening for nucleic acid sequences encoding a polypeptide comprising a signal sequence and/or a transmembrane sequence comprising: a) a replication system functional in a bacterial host cell; b) at least a first marker gene; and c) a candidate nucleic acid sequence; wherein expression of the marker gene in a bacterial cell indicates that the candidate nucleic acid sequence encodes a polypeptide comprising signal sequence and/or a transmembrane sequence.

In some embodiments, the first marker gene of the construct is a screenable marker gene, a scorable marker gene, a measurable marker gene or a selectable marker gene. In some specific aspects, the first marker gene is an antibiotic resistance gene and can be an ampicillin-resistance gene. In some aspects, the marker gene is mutated. In other aspects, the construct further comprises a multiple cloning site. In some embodiments, the host of the construct is a bacterial cell. The bacterial cell is a gram negative bacterial cell and may be an E. coli cell. Various E. coli strains are contemplated as useful and include, but are not limited to, MC1061, DH5a, Y1090 and JM101.

Also provided by the invention are proteins comprising signal sequences and/or transmembrane sequences from any eukaryotic cells. The present invention provides isolated polynucleotides encoding these proteins. Thus, the present invention provides isolated polynucleotide sequences or fragments thereof encoding for amino acid sequences of proteins comprising signal sequences and/or transmembrane sequences from any eukaryotic cells, determined by the methods of the present invention.

Some aspects of the invention also provide an isolated polynucleotide comprising a region having a sequence having at least 15 contiguous nucleotides in common with at least one nucleic acid sequence isolated from an eukaryotic cell or the complement of such a sequence. In other aspects, the isolated polynucleotides are further defined as comprising a sequence having least 50 contiguous nucleotides in common with at least one nucleic acid sequence isolated from an eukaryotic cell or the complement of such a sequence or the complement of such a sequence. In yet other aspects, the isolated polynucleotides are further defined as comprising a sequence having all nucleotides in common with at least one nucleic acid sequence isolated from an eukaryotic cell or the complement of such a sequence or the complement of such a sequence. Also provided are polypeptides from an eukaryotic cell having a region having an amino acid sequence determined by the methods of the present invention as described above or a fragment thereof. In some embodiments, the polypeptides are further defined as a recombinant polypeptides.

The invention also provides methods of producing a polypeptide having a region having an amino acid sequence determined by the methods of the present invention as described above or fragment thereof, comprising: a) obtaining a polynucleotide comprising a region encoding at least one nucleic acid sequence isolated from an eukaryotic cell or the complement of such a sequence or a fragment thereof, and b) expressing the polynucleotide to obtain the polypeptide.

In some embodiments of the methods, the polynucleotide has a region having a sequence of at least one nucleic acid sequence isolated from an eukaryotic cell or the complement of such a sequence or a fragment thereof.

The invention also provides antibodies directed against a polypeptide from eukaryotic cells having a region having an amino acid sequence determined by the methods of the present invention as described above, or an antigenic fragment thereof. The antibody can be a monoclonal antibody. Such antibodies could be used for either diagnostic or therapeutic purposes.

The invention also contemplates that other specific aspects of fat cell function may be assayed by using the nucleic acids and/or polypeptides identified by the screening methods of the present invention. These aspects of fat cell function include sugar and fat metabolism, insulin resistance, diabetes, hyperglycemia, hypoglycemia, and lipid abnormalities including conditions that lead to increased levels of cholesterol, triglycerides, LDL, etc.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Map of plasmid construct.

### DESCRIPTION OF ILLUSTRATIVE EMBODIMENTS

Identification of proteins comprising signal sequences and/or transmembrane sequences is important for medical diagnosis, as well as in research and industry, given the numerous applications that such proteins may be used in conjunction with. For example, novel diagnostic blood tests designed to screen for proteins that comprise a signal sequence and/or a transmembrane sequence can be developed to diagnose several diseases. Hormones comprise another important group of secreted factors and are of great therapeutic value, for example, insulin, leptin, etc. Identification of new hormones is thus another important facet of the present invention. In other examples, one may attach a strong signal sequence to a gene encoding a protein of interest to render a secreted protein which is easier to isolate and purify. In addition, proteins comprising signal sequences/transmembrane sequences are those involved in cell-signaling and signal transduction. Thus, they are potentially of great therapeutic value for purposes of drug discovery. Molecules that selectively modulate the function of such membrane-bound proteins have been found to be effective therapies for a wide variety of diseases and disorders. Membrane-bound proteins may also be suitable targets for the development of therapeutic antibodies. The existing methods to identify proteins comprising signal sequences and/or transmembrane sequences require extended screening procedures and are not very efficient.

The present invention provides simple and effective screening methods to identify nucleic acids that encode eukaryotic proteins comprising signal sequences and/or transmembrane sequences using methods based on bacterial screening. For the screening, the inventors have utilized a nucleic acid construct that expresses a marker gene that is expressed only if an intact signal sequence region is present in the construct. Therefore, constructs that comprise a mutation in the signal sequence region are used for the screening assays of the invention.

The marker gene contemplated of use includes any marker gene that requires a signal sequence for appropriate expression. Thus, the marker gene product is a gene that is typically a secreted or membrane bound protein. In one non-limiting example, the invention describes an ampicillin resistance marker gene which has a mutation in its signal sequence region. The present invention is exemplified by utilizing Escherichia coli (E. coli) as the host cell. E. coli are simple organisms that are easy to grow and manipulate, although other prokaryotic organisms are also contemplated as useful.

High-throughput screening methods are described for the rapid screening, identification and isolation of proteins comprising signal sequences and/or transmembrane sequences. Thus, the methods of this invention can be employed to identify signal sequences present in any DNA fragment, for example, from genomic DNA libraries, from cDNA libraries, oligonucleotide libraries, tissue-specific cDNA libraries, etc. Once positive clones are identified, they are subject to multi-well DNA isolation, multi-well amplification, microchip analysis, and extensive DNA sequencing for identification.

Utilizing the methods of the invention, numerous eukaryotic proteins comprising signal sequences and/or transmembrane sequences from breast cancers as well as from adipose tissues have been isolated. For example, several novel breast cancer proteins comprising transmembrane/signal sequences have been isolated and identified and are represented by the amino acid sequences set forth in SEQ ID NO: 18, SEQ ID NO: 24, SEQ ID NO: 28, SEQ ID NO: 38, SEQ ID NO: 44, SEQ ID NO: 48, SEQ ID NO: 54, SEQ ID NO: 72, SEQ ID NO: 74, SEQ ID NO: 76, SEQ ID NO: 78, SEQ ID NO: 84, SEQ ID NO: 86, SEQ ID NO: 92, SEQ ID NO: 94, SEQ ID NO: 98, SEQ ID NO: 100, SEQ ID NO: 104, SEQ ID NO: 110, SEQ ID NO: 112, SEQ ID NO: 126, SEQ ID NO: 130, which correspond to the nucleic acid sequences comprised in, SEQ ID NO: 17, SEQ ID NO: 23, SEQ ID NO: 27, SEQ ID NO: 37, SEQ ID NO: 43, SEQ ID NO: 47, SEQ ID NO: 53, SEQ ID NO: 71, SEQ ID NO: 73, SEQ ID NO: 75, SEQ ID NO: 77, SEQ ID NO: 83, SEQ ID NO: 85, SEQ ID NO: 91, SEQ ID NO: 93, SEQ ID NO: 97, SEQ ID NO: 99, SEQ ID NO: 103, SEQ ID NO: 109, SEQ ID NO: 111, SEQ ID NO: 125, SEQ ID NO: 129.

Other breast cancer proteins comprising transmembrane/signal sequences identified by the methods of the invention represent proteins that have previously been characterized but are not know to be markers of breast cancer and these are represented by the amino acid sequences set forth in SEQ ID NO: 4 (Testis enhanced gene transcript), SEQ ID NO: 8 (Initiation factor 4B), SEQ ID NO: 10 (GalNAc-T), SEQ ID NO: 14 (HNF3A), SEQ ID NO: 16 (DRPLA), SEQ ID NO: 20 (Nuclear receptor interacting protein 1), SEQ ID NO: 26 (Integral membrane protein 2B), SEQ ID NO: 30 (Amino acid transporter system A1), SEQ ID NO: 32 (Rab5b), SEQ ID NO: 34 (P4HA1), SEQ ID NO: 36 (LIV-1), SEQ ID NO: 40 (MAPK1), SEQ ID NO: 42 (Choline/ethanolamine phosphotransferase), SEQ ID NO: 50 (G3BP2 (KIAA0660)), SEQ ID NO: 52 (Beta actin), SEQ ID NO: 56 (Gamma actin), SEQ ID NO: 58 (13 kDa differentiation-associated protein/NADH Ubiquinone Oxidoreductase subunit B17.2), SEQ ID NO: 60 (SEL1L), SEQ ID NO: 62 (ATPase, ClassII, type 9A (KIAA0611)), SEQ ID NO: 64 (NHE3RF), SEQ ID NO: 66 (SLC7A2), SEQ ID NO: 68 (VDAC1), SEQ ID NO: 70 (PRG1), SEQ ID NO: 80 (ATPase beta 1 polypeptide), SEQ ID NO: 82 (Cyclophilin B), SEQ ID NO: 88 (Fibulin-1 isoform D precursor), SEQ ID NO: 96 (APG-1), SEQ ID NO: 102 (guanine nucleotide exchange factor), SEQ ID NO: 114 (Immunoglobulin gamma heavy chain), SEQ ID NO: 116 (KCNMB1), SEQ ID NO: 120 (Similar to sialyltransferase 7), SEQ ID NO: 122 (syntaxin binding protein 1), SEQ ID NO: 128 (Collagen I, alpha-1 polypeptide), the corresponding nucleic acid sequences being, SEQ ID NO: 3 (Testis enhanced gene transcript), SEQ ID NO: 7 (Initiation factor 4B), SEQ ID NO: 9 (GalNAc-T), SEQ ID NO: 13 (HNF3A), SEQ ID NO: 15 (DRPLA), SEQ ID NO: 19 (Nuclear receptor interacting protein 1), SEQ ID NO: 25 (Integral membrane protein 2B), SEQ ID NO: 29 (Amino acid transporter system A1), SEQ ID NO: 31 (Rab5b), SEQ ID NO: 33 (P4HA1), SEQ ID NO: 35 (LIV-1), SEQ ID NO: 39 (MAPK1), SEQ ID NO: 41 (Choline/ethanolamine phosphotransferase), SEQ ID NO: 49 (G3BP2 (KIAA0660)), SEQ ID NO: 51 (Beta actin), SEQ ID NO: 55 (Gamma actin), SEQ ID NO: 57 (13 kDa differentiation-associated protein/NADH Ubiquinone Oxidoreductase subunit B17.2), SEQ ID NO: 59 (SEL1L), SEQ ID NO: 61 (ATPase, ClassII, type 9A (KIAA0611)), SEQ ID NO: 63 (NHE3RF), SEQ ID NO: 65 (SLC7A2), SEQ ID NO: 67 (VDAC1), SEQ ID NO: 69 (PRG1), SEQ ID NO: 79 (ATPase beta 1 polypeptide), SEQ ID NO: 81 (Cyclophilin B), SEQ ID NO: 87 (Fibulin-1 isoform D precursor), SEQ ID NO: 95 (APG-1), SEQ ID NO: 101 (guanine nucleotide exchange factor), SEQ ID NO: 113 (Immunoglobulin gamma heavy chain), SEQ ID NO: 115 (KCNMB 1), SEQ ID NO: 119 (Similar to sialyltransferase 7), SEQ ID NO: 121 (syntaxin binding protein 1), SEQ ID NO: 127 (Collagen I, alpha-1 polypeptide).

Still other breast cancer proteins comprising transmembrane/signal sequences identified by the methods of the invention represent proteins that have previously been characterized as markers of breast cancer and these are represented by the amino acid sequences set forth in SEQ ID NO: 2 (CD9 antigen), SEQ ID NO: 6 (Prothymosin alpha), SEQ ID NO: 12 (IGFBP5), SEQ ID NO: 22 (KAP1), SEQ ID NO: 46 (Claudin 7), SEQ ID NO: 90 (Transferrin receptor), SEQ ID NO: 106 (IGFBP7), SEQ ID NO: 108 (Fibronectin), SEQ ID NO: 118 (SPARC/Osteonectin), SEQ ID NO: 124 (Osteopontin), the corresponding nucleic acid sequences being SEQ ID NO: 1 (CD9 antigen), SEQ ID NO: 5 (Prothymosin alpha), SEQ ID NO: 11 (IGFBP5), SEQ ID NO: 21 (KAP1), SEQ ID NO: 45 (Claudin 7), SEQ ID NO: 89 (Transferrin receptor), SEQ ID NO: 105 (IGFBP7), SEQ ID NO: 107 (Fibronectin), SEQ ID NO: 117 (SPARC/Osteonectin), SEQ ID NO: 123 (Osteopontin).

The inventors have also identified several novel proteins comprising transmembrane and/or signal sequences from adipocyte (fat) cells and these are represented by the amino acid sequences SEQ ID NO: 135, SEQ ID NO: 140, SEQ ID NO: 142, SEQ ID NO: 145, SEQ ID NO: 157, SEQ ID NO: 159, SEQ ID NO: 161, SEQ ID NO: 163, SEQ ID NO: 172, SEQ ID NO: 174, SEQ ID NO: 176, SEQ ID NO: 182, SEQ ID NO: 188, SEQ ID NO: 190, SEQ ID NO: 199, SEQ ID NO: 201, SEQ ID NO: 210, SEQ ID NO: 214, SEQ ID NO: 218, SEQ ID NO: 234, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 258, SEQ ID NO: 266, SEQ ID NO: 268, SEQ ID NO: 270, SEQ ID NO: 278, SEQ ID NO: 280, SEQ ID NO: 286, SEQ ID NO: 288, SEQ ID NO: 297. These and other novel proteins comprising transmembrane and/or signal sequences from adipocyte (fat) cells are represented by the nucleic acid sequences comprised in SEQ ID NO: 134, SEQ ID NO: 138, SEQ ID NO: 139, SEQ ID NO: 141, SEQ ID NO: 143, SEQ ID NO: 144, SEQ ID NO: 151, SEQ ID NO: 156, SEQ ID NO: 158, SEQ ID NO: 160, SEQ ID NO: 162, SEQ ID NO: 171, SEQ ID NO: 173, SEQ ID NO: 175, SEQ ID NO: 181, SEQ ID NO: 187, SEQ ID NO: 189, SEQ ID NO: 198, SEQ ID NO: 200, SEQ ID NO: 208, SEQ ID NO: 209, SEQ ID NO: 213, SEQ ID NO: 217, SEQ ID NO: 233, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 257, SEQ ID NO: 265, SEQ ID NO: 267, SEQ ID NO: 269, SEQ ID NO: 277, SEQ ID NO: 279, SEQ ID NO: 285, SEQ ID NO: 287, SEQ ID NO: 296, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324.

Other proteins comprising transmembrane and/or signal sequences isolated by the methods of the present invention from adipocyte (fat) cells which have previously been characterized but have not been found before in fat/adipocyte cells are represented by the amino acid sequences comprised in SEQ ID NO: 132 (mFizz1), SEQ ID NO: 147 (per-pentamer repeat gene), SEQ ID NO: 150 (PCAP 5′UTR), SEQ ID NO: 165 (SOX9), SEQ ID NO: 166 (Adenylate cyclase 6), SEQ ID NO: 168 (TTS-2 transport secretion protein), SEQ ID NO: 170 (guanine nucleotide binding protein, gamma 11), SEQ ID NO: 176 (junctional adhesion molecule precursor), SEQ ID NO: 192 (lectin B), SEQ ID NO: 197 (Mac-1, CD11b), SEQ ID NO: 238 (amyloid beta (A4) precursor-like protein), SEQ ID NO: 240 (macrophage maturation-associated transcript dd3f protein), SEQ ID NO: 256 (decorin), SEQ ID NO: 276 (CD39 antigen), SEQ ID NO: 295 (CD94: NKG2D natural killer cell receptor (lectin)). Nucleic acid sequences corresponding to these and other proteins comprising transmembrane and/or signal sequences isolated by the methods of the present invention from adipocyte (fat) cells which have previously been characterized but have not been reported in fat/adipocyte cells are represented by SEQ ID NO: 131 (mFizz1), SEQ ID NO: 146 (per-pentamer repeat gene), SEQ ID NO: 148 (osteoclast stimulating factor 1), SEQ ID NO: 149 (PCAP 5′UTR), SEQ ID NO: 164 (SOX9), SEQ ID NO: 167 (TTS-2 transport secretion protein), SEQ ID NO: 169 (guanine nucleotide binding protein, gamma 11), SEQ ID NO: 175 (junctional adhesion molecule precursor), SEQ ID NO: 191 (lectin B), SEQ ID NO: 196 (Mac-1, CD11b), SEQ ID NO: 237 (amyloid beta (A4) precursor-like protein), SEQ ID NO: 239 (macrophage maturation-associated transcript dd3f protein), SEQ ID NO: 255 (decorin), SEQ ID NO: 275 (CD39 antigen), SEQ ID NO: 294 (CD94: NKG2D natural killer cell receptor (lectin)), SEQ ID NO: 320 (homology to macrophage galactose N-acetylgalacotsamine-specific lectin).

Still other fat sequences that have been sequenced, but not subject to identification as to being novel or previously characterized, and are represented by the amino acid sequences in SEQ ID NO: 137, SEQ ID NO: 155, SEQ ID NO: 178, SEQ ID NO: 180, SEQ ID NO: 184, SEQ ID NO: 186, SEQ ID NO: 194, SEQ ID NO: 205, SEQ ID NO: 207, SEQ ID NO: 212, SEQ ID NO: 216, SEQ ID NO: 220, SEQ ID NO: 222, SEQ ID NO: 224, SEQ ID NO: 226, SEQ ID NO: 228, SEQ ID NO: 230, SEQ ID NO: 232, SEQ ID NO: 236, SEQ ID NO: 260, SEQ ID NO: 262, SEQ ID NO: 264, SEQ ID NO: 274, SEQ ID NO: 282, SEQ ID NO: 284, SEQ ID NO: 290, SEQ ID NO: 293, SEQ ID NO: 299 and the nucleic acids comprised in SEQ ID NO: 133, SEQ ID NO: 136, SEQ ID NO: 154, SEQ ID NO: 177, SEQ ID NO: 179, SEQ ID NO: 183, SEQ ID NO: 185, SEQ ID NO: 193, SEQ ID NO: 195, SEQ ID NO: 204, SEQ ID NO: 206, SEQ ID NO: 211, SEQ ID NO: 215, SEQ ID NO: 219, SEQ ID NO: 221, SEQ ID NO: 223, SEQ ID NO: 225, SEQ ID NO: 227, SEQ ID NO: 229, SEQ ID NO: 231, SEQ ID NO: 235, SEQ ID NO: 259, SEQ ID NO: 261, SEQ ID NO: 263, SEQ ID NO: 273, SEQ ID NO: 281, SEQ ID NO: 283, SEQ ID NO: 289, SEQ ID NO: 291, SEQ ID NO: 292.

The inventors also contemplate identifying differentially expressed proteins and nucleic acids in biologically meaningful situations. For example, identifying proteins comprising signal sequences and/or transmembrane sequences expressed only in breast cancer cells, and not in normal breast tissue, allows the use of such proteins in developing diagnostic/prognostic detection protocols for breast cancer. In another example, identifying proteins comprising signal sequences and/or transmembrane sequences expressed in fibroblasts versus adipocytes, or in lean animals versus obese animals, etc., allows for the identification of key proteins involved in fat metabolism. Thus, the inventors contemplate utilizing these methods for identifying key proteins in disease pathways, physiologic, and abnormal conditions.

A. Breast Cancer

Cancer has become one of the leading causes of death in the western world, second only behind heart disease. Current estimates project that one person in three in the U.S. will develop cancer, and that one person in five will die from cancer. Breast cancer is the most common cancer among women. The American Cancer Society estimates that in 2001 about 192,200 new cases of invasive breast cancer (Stages I-IV) will be diagnosed among women in the United States. Breast cancer also occurs in men and an estimated 1,500 cases will be diagnosed among men. In 2001, it is estimated that there will be about 40,600 deaths from breast cancer in the United States (40,200 among women, and 400 among men). Breast cancer is the second leading cause of cancer death in women, exceeded only by lung cancer.

Major challenges remain to be overcome for all cancers and this makes it essential to uncover the different molecular processes that lead to cancer and also identify protein markers that are expressed by cells during carcinogenesis. Identification of novel breast cancer proteins as well as other molecular players that are involved in the onset and progress of the cancer will ultimately lead to better and earlier detection protocols and improved treatment. Cancer markers are proteins that are generally in the cell membrane and comprise signal sequences.

B. Fat Metabolism

The ability to store energy, primarily as fat, is required for the life cycle of higher organisms. Unfortunately, modern life has generated negative consequences of fat storage, obesity. There has been a dramatic worldwide increase in the prevalence of obesity to the point where the majority of adults in America and Europe are considered overweight. Notably, obesity leads to decreased survival as it is associated with the development of many diseases, most notably type II diabetes mellitus, coronary artery disease, hypertension, sleep apnea, arthritis, and even some cancers. In the US alone, estimates indicate that approximately 300,000 people die annually from obesity at a financial cost of more than 100 billion dollars. Globally, over a billion people suffer negative health consequences from excess weight, which is replacing malnutrition and infectious diseases as the most significant cause of illness throughout the world. Therefore, identifying molecules that can alter the ability to store fat has widespread ramifications.

Historically, the adipocyte has been thought of as a passive conduit i.e., reflecting the amount of food consumed by an organism. However, recent evidence demonstrates that fat storage is under dynamic control and several proteins and hormones are involved in fat metabolism. For example, signals are received on the adipocyte (fat cell) to regulate its actions. In return the adipocyte sends signals, such as a leptin, to other parts of the body to control fat accumulation (Friedman et al., 1998). Recently, another adipocyte-secreted hormone, resistin, was described which was indicated to be a link between obesity and diabetes. For example, blocking resistin function improved blood glucose and insulin resistance in mice with diet-induced obesity (Steppan et al., 2001). Therefore, it seems likely that discovering additional adipocyte-secreted signals may offer potential benefits to the millions of people affected by obesity and diabetes.

C. Vectors of the Invention

The invention also provides plasmid vectors that have been designed to identify DNA sequences comprising signal sequences. These vectors allow screening of genomic DNA fragments or cDNA fragments for the presence of signal sequences. The DNA fragments are usually unidentified fragments. The vectors of the invention are characterized by having a plurality of functional sequences.

Origin of Replication. The vectors of the invention have at least one origin of replication. In order to propagate a vector in a host cell, it may contain one or more origins of replication (often termed “ori”), which is a specific nucleic acid sequence at which replication is initiated. Alternatively an autonomously replicating sequence (ARS) can be employed if the host cell is yeast. Suitable origins of replication include, for example, the ColE1, pSC101 and M13 origins of replication.

Promoters. A “promoter” is a control sequence that is a region of a nucleic acid sequence at which initiation and rate of transcription are controlled. It may contain genetic elements on which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

The vectors of the invention, optionally has one or more promoters. The presence of the promoter allows for detection of signal sequences which have been separated from their wild-type promoter. Thus, relatively small DNA fragments may be screened and the presence of the signal sequences detected.

A promoter generally comprises a sequence that functions to position the start site for RNA synthesis. The best known example of this is the TATA box. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference).

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, cell-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment.

Cloning Site. Another optional functional element that can comprise the vectors of the invention is a cloning site. Cloning sites contain at least one restriction enzyme site, which can be used in conjunction with standard recombinant technology to digest the vector (see, for example, Carbonelli et al., 1999, Levenson et al., 1998, and Cocea, 1997, incorporated herein by reference). One example of a cloning site is a multiple cloning site (MCS). An MCS is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference). An MCS is characterized by having at least two, usually at least three, and as many as ten, restriction sites, at least two of which, and preferably all, are unique to the vector. Thus, the vector will be capable of being cleaved uniquely in the MCS. The cloning sites may be blunt ended or have overhangs of from 1 to many nucleotides. Restriction enzymes with overhangs are preferred. The overhangs will be capable of both, hybridizing with the overhangs obtained with restriction enzymes other than the restriction enzyme which cleaves at the restriction site in the MCS, and hybridizing with the overhangs obtained with the same restriction enzyme.

The MCS will usually be not more than about 100 nucleotides, usually not more than about 60 nucleotides, and generally at least about 40 nucleotides, and more usually at least about 20 nucleotides. The MCS will also be free of stop codons in the translational reading frame for the structural genes. Where a convenient MCS is commercially available, the MCS may be modified by cleavage at a restriction site in the MCS and removal or addition of a number of nucleotides other than 3 or a multiple of 3. The MCS may provide a chain of two of more amino acids between the genomic fragment and the expression product. Usually, the MCS will provide fewer than 30 amino acids, preferably fewer than about 20 amino acids. Of course, the number of amino acids introduced by the MCS will depend not only upon the size of the MCS, but also the site at which the DNA fragment is inserted into the MCS.

Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Marker Gene. The marker gene, which is employed, can be any gene that in addition to being readily detected requires a functional signal sequence for appropriate expression. In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, an antibiotic resistance gene, such as genes that confer resistance to ampicillin, kanamycin, neomycin, puromycin, hygromycin, zeocin, tetracycline HAT, and histidinol are useful selectable markers. In other examples, multidrug resistance genes, herbicide resistance genes, or toxin resistance genes may be useful as a selectable marker. In addition to markers conferring a phenotype that allows for the discrimination of transformants based on the implementation of conditions, other types of markers including screenable markers such as a fluorescent protein gene (such as, a green fluorescent protein (GFP), a yellow fluorescent protein, a blue fluorescent protein, or a red fluorescent protein), whose basis is fluorimetric analysis, are also contemplated. Alternatively, screenable enzymes such as lac z or beta-galactosidase may be utilized. One could also use a selectable marker gene that allows for selection on media deficient in certain nutrients. Examples of such markers include a DHFR gene and HAT gene.

The marker may be a scorable marker gene, a measurable marker gene, or a selectable marker. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable, screenable and scorable markers are well known to one of skill in the art.

For detection, the marker gene product generally confers resistance to an antibiotic, or requires a specific metabolite for the host cell to grow, or other means which allows for rapid screening of secretion of the expression product. In context of the vectors of the present invention, an ampicillin resistance gene, a penicillin-resistance gene, a cephalosporin-resistance gene, an oxacephem-resistance gene, a carbapenem-resistance gene, or a monobactam-resistance gene may be used.

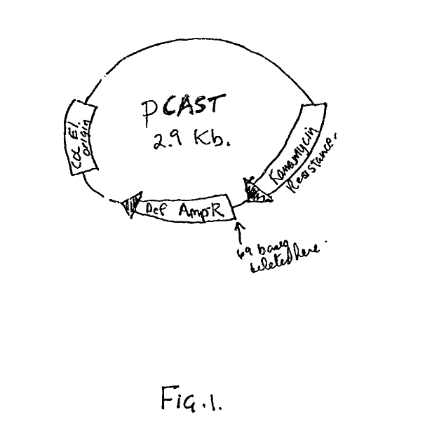

peCAST. In carrying out the subject invention, one of the vectors prepared is a plasmid based vector, peCAST. peCAST is shown in FIG. 1. This vector was constructed using the plasmid pCRII-TOPO (Invitrogen, San Diego, Calif.). A sixty-nine nucleotide deletion at the extreme 5′-end of the ampicillin-resistance (Amp-R) was generated, which corresponds to 23 amino acids at the amino-terminal that begin at the starting methionine and comprise the native signal sequence that targets the Amp-R gene product to the extracellular space in the bacteria. A 20-base multiple cloning site was cloned in place of this 69-base deletion.

In a non-limiting example, E. coli is often transformed using derivatives of peCAST. peCAST contains genes for kanamycin resistance and thus provides easy means for identifying transformed cells. The peCAST plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

D. Signal Peptides/Sequences

Signal peptides, also known as signal sequences or leader sequences, comprise a short amino-terminal sequence that is present in the initial version of newly translated secreted proteins or transmembrane proteins. This sequence targets these proteins to specialized cellular secretory pathways by initially targeting these proteins to cellular compartments that process such proteins including the endoplasmic reticulum.

The signal peptide or signal sequence comprises several elements necessary for targeting, the most important being a hydrophobic component. Immediately preceding the hydrophobic sequence there are often one or more basic amino acid(s), and at the carboxyl-terminal end of the signal peptide there generally are a pair of small, uncharged amino acids separated by a single intervening amino acid which is the site of cleavage by a signal peptidase. Although, the hydrophobic component, basic amino acid and peptidase cleavage site can usually be identified in the signal peptide of many known secreted proteins, the high level of degeneracy in any one of these elements makes difficult the identification or isolation of secreted or transmembrane proteins solely by hybridization with DNA probes designed to recognize cDNA's encoding signal peptides.

Secreted and membrane-bound cellular proteins have wide applicability in various industrial applications, including pharmaceuticals, diagnostics, biosensors and bioreactors. For example, many protein drugs commercially available at present, such as thrombolytic agents, interferons, interleukins, erythropoietins, colony stimulating factors, and various other cytokines are secretory proteins. Their receptors, which are membrane proteins, also have potential as therapeutic or diagnostic agents and most drugs are targetted to cell surface proteins. Thus, there is need to identify novel proteins that have signal sequences.

E. Gene Constructs

The nucleic acids used in the present invention may be prepared by recombinant nucleic acid methods. To express a DNA sequence, such as candidate DNA fragments and sequences that comprise a signal sequence, transcriptional and translational signals recognized by an appropriate host are necessary. A wide variety of transcriptional and translational regulatory sequences may be employed, depending upon the nature of the host. Transcriptional initiation regulatory signals may be selected that allow for repression or activation, so that expression of the genes can be modulated. One such controllable modulation technique is the use of regulatory signals that are temperature-sensitive, so that expression can be repressed or initiated by changing the temperature. Another controllable modulation technique is the use of regulatory signals that are sensitive to certain chemicals.

Expression Vectors. The term “expression vector” refers to any type of genetic construct comprising a nucleic acid coding for an RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described supra.

Expression vehicles for production of the molecules of the invention include plasmids or other vectors. In general, such vectors contain control sequences that allow expression in various types of hosts, including prokaryotes. Suitable expression vectors containing the desired coding and control sequences may be constructed using standard recombinant DNA techniques known in the art, many of which are described in Sambrook et al. (1989), Molecular Cloning: A Laboratory Manual, Second Edition, Cold Spring Harbor Laboratory, Cold Spring Habor, N.Y.

Expression vectors useful in the present invention typically contain an origin of replication. Suitable origins of replication include the colE1 origin of replication. The vectors may also optionally include a promoter located 5′ to (i.e., upstream of) the DNA sequence to be expressed, and a transcription termination sequence. The optional promoter sequence may also be inducible, to allow modulation of expression (e.g., by the presence or absence of nutrients or other inducers in the growth medium). One example is the lac operon obtained from bacteriophage lambda, which can be induced by IPTG.

The expression vectors may also include other regulatory sequences for optimal expression of the desired product. Such sequences include sequences that provide for stability of the expression product; enhancer sequences, which upregulate the expression of the DNA sequence; and restriction enzyme recognition sequences, which provide sites for cleavage by restriction endonucleases. All of these materials are known in the art and are commercially available.

In expression, one will typically include a polyadenylation signal to effect proper polyadenylation of the transcript. The nature of the polyadenylation signal is not believed to be crucial to the successful practice of the invention, and any such sequence may be employed. Polyadenylation may increase the stability of the transcript or may facilitate cytoplasmic transport.

A suitable expression vector may also include marker sequences, which allow phenotypic selection of transformed host cells. Such a marker may provide prototrophy to an auxotrophic host, antibiotic resistance and the like. The selectable marker gene can either be directly linked to the DNA gene sequences to be expressed, or introduced into the same cell by co-transfection. Examples of selectable markers include kanamycin, neomycin, ampicillin, hygromycin resistance and the like.

DNA Fragments. Candidate DNA sequences that comprise a signal sequence/transmembrane sequence may be obtained from a variety of sources, including from genomic DNA, subgenomic DNA, cDNA and libraries thereof. Genomic and cDNA libraries may be obtained in a number of ways as are known to the skilled artisan. Cells coding for the desired sequence may be isolated, the genomic DNA fragmented, for example, by treatment with one or more restriction endonucleases, and the resulting fragments cloned.

For preparation of cDNA, mRNA is isolated and reverse transcription is used to synthesize the second strand. Methods for reverse transcription and synthesis of cDNA are well known to the skilled artisan and are described in Sambrook et al. (1989), Molecular Cloning: A Laboratory Manual, Second Edition, Cold Spring Harbor Laboratory, Cold Spring Habor, N.Y.

Genomic DNA fragments may be screened by obtaining either a genomic library, which is a collection of DNA fragments obtained by digesting chromosomal or genomic DNA with one or more of a restriction endonuclease, or an endonuclease, or may even be DNA fragments from sheared chromosomal DNA.

In a non-limiting example, the DNA fragments which are employed will usually be at least about 10 to about 14, or about 15, about 20, about 30, about 40, about 50, about 100, about 200, about 500, about 1,000, about 2,000, about 3,000, about 5,000, about 10,000, about 15,000, about 20,000, about 30,000, about 50,000, about 100,000, about 250,000, about 500,000, about 750,000, to about 1,000,000 nucleotides in length, as well as constructs of greater size, up to and including chromosomal sizes (including all intermediate lengths and intermediate ranges), given the advent of nucleic acids constructs such as a yeast artificial chromosome are known to those of ordinary skill in the art. It will be readily understood that “intermediate lengths” and “intermediate ranges”, as used herein, means any length or range including or between the quoted values (i.e., all integers including and between such values). Non-limiting examples of intermediate lengths include about 11, about 12, about 13, about 16, about 17, about 18, about 19, etc.; about 21, about 22, about 23, etc.; about 31, about 32, etc.; about 51, about 52, about 53, etc.; about 101, about 102, about 103, etc.; about 151, about 152, about 153, etc.; about 1,001, about 1002, etc,; about 50,001, about 50,002, etc; about 750,001, about 750,002, etc.; about 1,000,001, about 1,000,002, etc. Non-limiting examples of intermediate ranges include about 3 to about 32, about 150 to about 500,001, about 3,032 to about 7,145, about 5,000 to about 15,000, about 20,007 to about 1,000,003, etc.

Various techniques can be employed to control the size of the fragment. For example, one can use a restriction endonuclease providing a complementary overhang 1 5 and a second restriction endonuclease to recognize a relatively common site, but provides a terminus which is not complementary to the terminus of the vector restriction site.

After joining the fragments to the cleaved vector, one may further subject the resulting linear DNA to additional restriction enzymes, where the vector lacks recognition sites for such restriction enzymes. In this way, a variety of sizes can be obtained.

F. Identification

Clones which comprise DNA sequences with signal sequences can be further analyzed in a variety of ways. The insert can be excised, using the flanking restriction sites, either those employed for insertion or those present in the MCS and the resulting fragment can be isolated. This fragment can also be sequenced, either directly from the construct/plasmid or by synthesizing fragments by PCR™ from the construct/plasmid so that the initiation codon and signal sequence is determined. Additionally, the protein product may be sequenced to determine the site at which processing occurred. The nucleic acid sequence can also be used as a probe to determine the wild-type gene which employs the particular signal sequence. Thus, the DNA sequence corresponding to the gene that comprises the signal sequence can be isolated.

G. Microarray/Chip Technologies

Specifically contemplated by the present inventors are microarray or chip-based DNA technologies such as those described by Hacia et al. (1996) and Shoemaker et al. (1996). These techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (Pease et al., 1994; Fodor et al., 1991. The present inventors envision that peCAST positive clones will be used to generate PCR fragments to generate a microchip array.

H. Nucleic Acid Detection

A variety of nucleic acid detection and/or amplification techniques are suitable for use with the probes and primers that comprise the nucleic acid sequences provided by the present invention in methods for detecting the presence of cancer markers or other proteins comprising a signal- and/or a transmembrane-sequence in a biological sample.

These embodiments of the invention comprise methods for the identification of cancer cells in biological samples by detecting nucleic acids that correspond to cancer cell markers and are not present in normal cells. The biological sample can be any tissue or fluid in which the cancer cells might have secreted or transmembrane cancer marker protein comprising a signal-sequence. Alternatively, the biological sample can be any tissue or fluid in which the cancer cells might have metastasized to and thus one can detect a cancer marker protein that comprises a transmembrane or secreted sequence.

Tissue sections, specimens, aspirates and biopsies also may be used. Further suitable examples are bone marrow aspirates, bone marrow biopsies, spleen tissues, fine needle aspirates and even skin biopsies. Other suitable examples are fluids, including samples where the body fluid is peripheral blood, serum, lymph fluid, seminal fluid or urine. Stools may even be used.

The nucleic acids, used as a template for detection, are isolated from cells contained in the biological sample, according to standard methodologies (Sambrook et al., 1989). The nucleic acid may be genomic DNA or fractionated or whole cell RNA.

Northern Blotting. In certain embodiments, RNA detection is by Northern blotting, i.e., hybridization with a labeled probe. The techniques involved in Northern blotting are well known to those of skill in the art and can be found in many standard books on molecular protocols (e.g., Sambrook et al., 1989).

Briefly, RNA is separated by gel electrophoresis. The gel is then contacted with a membrane, such as nitrocellulose, permitting transfer of the nucleic acid and non-covalent binding. Subsequently, the membrane is incubated with, e.g., a labeled probe that is capable of hybridizing with a target amplification product. Detection is by exposure of the membrane to x-ray film, ion-emitting detection devices or colorimetric assays.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Reverse Transcriptase PCR™. In other embodiments, RNA detection can be performed using a reverse transcriptase PCR amplification procedure. Methods of reverse transcribing RNA into cDNA using the enzyme reverse transcriptase are well known and described in Sambrook et al., 1989. Alternative methods for reverse transcription utilize thermostable DNA polymerases. These methods are described in WO 90/07641.

I. Amplification and Detection

PCR. In one detection embodiment, DNA is used directly as a template for PCR amplification. In PCR, pairs of primers that selectively hybridize to nucleic acids corresponding to cancer-specific markers are used under conditions that permit selective hybridization. The term primer, as used herein, encompasses any nucleic acid that is capable of priming the synthesis of a nascent nucleic acid in a template-dependent process. Typically, primers are oligonucleotides from ten to twenty-five base pairs in length, but longer sequences can be employed. Primers may be provided in double-stranded or single-stranded form, although the single-stranded form is preferred.

The primers are used in any one of a number of template-dependent processes to amplify the marker sequences present in a given template sample. One of the best known amplification methods is the polymerase chain reaction (referred to as PCR) which is described in detail in U.S. Pat. No. 4,683,195, 4,683,202 and 4,800,159, each incorporated herein by reference, and in Innis et al. (1990, incorporated herein by reference).

In PCR, two primer sequences are prepared which are complementary to regions on opposite complementary strands of the cancer marker sequence. The primers will hybridize to form a nucleic acid:primer complex if the cancer marker sequence is present in a sample. An excess of deoxynucleoside triphosphates are added to a reaction mixture along with a DNA polymerase, e.g., Taq polymerase, that facilitates template-dependent nucleic acid synthesis.

If the marker sequence:primer complex has been formed, the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated. These multiple rounds of amplification, referred to as “cycles”, are conducted until a sufficient amount of amplification product is produced.

Next, the amplification product is detected. In certain applications, the detection may be performed by visual means. Alternatively, the detection may involve indirect identification of the product via chemiluminescence, electroluminescence, radioactive scintigraphy of incorporated radiolabel or fluorescent label or even via a system using electrical or thermal impulse signals (Affymax technology).

A reverse transcriptase PCR amplification procedure may be performed in order to quantify the amount of mRNA amplified. Methods of reverse transcribing RNA into cDNA are well known and described in Sambrook et al., 1989. Alternative methods for reverse transcription utilize thermostable DNA polymerases. These methods are described in WO 90/07641, filed Dec. 21, 1990.

Other Amplification Techniques. Another method for amplification is the ligase chain reaction (“LCR”), disclosed in European Patent Application No. 320,308, incorporated herein by reference. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750, incorporated herein by reference, describes a method similar to LCR for binding probe pairs to a target sequence.

Qbeta Replicase, described in PCT Patent Application No. PCT/US87/00880, also may be used as still another amplification method in the present invention. In this method, a replicative sequence of RNA which has a region complementary to that of a target is added to a sample in the presence of an RNA polymerase. The polymerase will copy the replicative sequence which can then be detected.

An isothermal amplification method, in which restriction endonucleases and ligases are used to achieve the amplification of target molecules that contain nucleotide 5-[-thio]-triphosphates in one strand of a restriction site also may be useful in the amplification of nucleic acids in the present invention. Such an amplification method is described by Walker et al. (1992, incorporated herein by reference).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA.

Target specific sequences can also be detected using a cyclic probe reaction (CPR). In CPR, a probe having 3 and 5 sequences of non-specific DNA and a middle sequence of specific RNA is hybridized to DNA which is present in a sample. Upon hybridization, the reaction is treated with RNase H, and the products of the probe identified as distinctive products which are released after digestion. The original template is annealed to another cycling probe and the reaction is repeated.

Other amplification methods, as described in British Patent Application No. GB 2,202,328, and in PCT Patent Application No. PCT/US89/01025, each incorporated herein by reference, may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; PCT Patent Application WO 88/10315, each incorporated herein by reference).

In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once against with a polymerase such as T7 or SP6. The resulting products, whether truncated or complete, indicate target specific sequences.

Davey et al., European Patent Application No. 329,822 (incorporated herein by reference) disclose a nucleic acid amplification process involving cyclically synthesizing single-stranded RNA (“ssRNA”), ssDNA, and double-stranded DNA (dsDNA), which may be used in accordance with the present invention.

The ssRNA is a first template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a second template for a second primer, which also includes the sequences of an RNA polymerase promoter (exemplified by T7 RNA polymerase) 5 to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Patent Application WO 89/06700 (incorporated herein by reference) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts.

Other suitable amplification methods include “race” and “one-sided PCR” (Frohman, 1990; Ohara et al., 1989, each herein incorporated by reference). Methods based on ligation of two (or more) oligonucleotides in the presence of nucleic acid having the sequence of the resulting “di-oligonucleotide”, thereby amplifying the di-oligonucleotide, also may be used in the amplification step of the present invention (Wu et al., 1989, incorporated herein by reference).

Separation Methods. Following amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982). In yet another alternative, labeled cDNA products, such as biotin or antigen can be captured with beads bearing avidin or antibody, respectively.

Identification Methods. Amplification products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled. In another embodiment, the probe is conjugated to a binding partner, such as an antibody or biotin, where the other member of the binding pair carries a detectable moiety.

J. Antibodies

Antibody Generation. The present invention contemplates the use of antibodies generated against some of the peptides/polypeptides/proteins comprising a signal sequence and/or a transmembrane domain identified by the methods of the invention. It is contemplated that the methods of the invention will identify several novel peptides/polypeptides/proteins comprising a signal sequence and/or a transmembrane domain and that some of these peptides/polypeptides/proteins will be disease markers. For example, several of the breast cancer peptides/polypeptides/proteins identified by the inventors are putative breast cancer markers that are found expressed solely or predominantly in cancers and are absent or found only at greatly reduced levels in normal breast tissues. Generation of antibodies to such marker peptides/polypeptides/proteins allows the rapid identification of the peptide/polypeptide/protein in a diagnostic assay. Alternatively, such antibodies could be used as therapeutic agents, either in modified or unmodified form. Thus, the generation of antibodies to the various peptides/polypeptides/proteins identified by the invention is another contemplated embodiment of the invention.

Means for preparing and characterizing antibodies are well known in the art (See, e.g., Antibodies: A Laboratory Manual, Cold Spring Harbor Laboratory, 1988; incorporated herein by reference). This section presents a brief discussion on the methods for generating antibodies.

Polyclonal Antibodies. Briefly, a polyclonal antibody is prepared by immunizing an animal with an immunogenic composition in accordance with the present invention and collecting antisera from that immunized animal.

A wide range of animal species can be used for the production of antisera. Typically the animal used for production of anti-antisera is a rabbit, a mouse, a rat, a hamster, a guinea pig or a goat. Because of the relatively large blood volume of rabbits, a rabbit is a preferred choice for production of polyclonal antibodies.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other proteins such as ovalbumin, mouse serum albumin, rabbit serum albumin, bovine thyroglobulin, or soybean trypsin inhibitor can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. Other bifunctional or derivatizing agent may also be used for linking, for example maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization.

A second, booster injection, also may be given. The process of boosting and titering is repeated until a suitable titer is achieved. When a desired level of immunogenicity is obtained, the immunized animal can be bled and the serum isolated and stored, and/or the animal can be used to generate monoclonal antibodies (mAbs).

For production of rabbit polyclonal antibodies, the animal can be bled through an ear vein or alternatively by cardiac puncture. The procured blood is allowed to coagulate and then centrifuged to separate serum components from whole cells and blood clots. The serum may be used as is for various applications or else the desired antibody fraction may be purified by well-known methods, such as affinity chromatography using another antibody or a peptide bound to a solid matrix or protein A followed by antigen (peptide) affinity column for purification.

Monoclonal Antibodies. A “monoclonal antibody” (mAbs), refers to homogenous populations of immunoglobulins which are capable of specifically binding to a peptides/polypeptides/proteins. It is understood that a given peptides/polypeptides/protein may have one or more antigenic determinants. The antibodies of the invention may be directed against one or more of these determinants.

Monoclonal antibodies (mAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified antigen protein, polypeptide or peptide. The immunizing composition is administered in a manner effective to stimulate antibody producing cells.

The methods for generating mAbs generally begin along the same lines as those for preparing polyclonal antibodies. Rodents such as mice and rats are preferred animals, however, the use of rabbit, sheep, goat, monkey cells also is possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen, generally as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B lymphocytes (B-cells), are selected for use in the mAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984; each incorporated herein by reference). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions.

One preferred murine myeloma cell is the NS-1 myeloma cell line (also termed P3-NS-1-Ag4-1), which is readily available from the NIGMS Human Genetic Mutant-cell Repository by requesting cell line repository number GM3573. Another mouse myeloma cell line that may be used is the 8-azaguanine-resistant mouse murine myeloma SP2/0 non-producer cell line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fission of cell membranes. Fusion methods using Sendai virus have been described by Kohler and Milstein (1975; 1976), and those using polyethylene glycol (PEG), such as 37% (v/v)

PEG, by Gefter et al. (1977). The use of electrically induced fusion methods also is appropriate (Goding pp. 71-74, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (hypoxanthine-aminopterin-thymidine (HAT) medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific mAb produced by the fused cell hybrid. The body fluids of the animal, such as serum or ascites fluid, can then be tapped to provide mAbs in high concentration.

The individual cell lines could also be cultured in vitro, where the mAbs are naturally secreted into the culture medium from which they can be readily obtained in high concentrations.

mAbs produced by either means may be further purified, if desired, using filtration, centrifugation and various chromatographic methods such as HPLC or affinity chromatography. Fragments of the mAbs of the invention can be obtained from the purified mAbs by methods which include digestion with enzymes, such as pepsin or papain, and/or by cleavage of disulfide bonds by chemical reduction. Alternatively, mAb fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobin preparations and U.S. Pat. No. 4,867,973 which describes antibody-therapeutic agent conjugates.

Humanized Antibodies. U.S. Pat. No. 5,565,332 describes methods for the production of antibodies, or antibody fragments, which have the same binding specificity as a parent antibody but which have increased human characteristics. Human mAbs can be made by the hybridoma method. Human myeloma and mouse-human heteromyeloma cell lines for the production of human mAbs have been described, for example, by Kozbor (1984), and Brodeur et al. (1987). Humanized antibodies may also be obtained by chain shuffling, perhaps using phage display technology, in as much as such methods will be useful in the present invention the entire text of U.S. Pat. No. 5,565,332 is incorporated herein by reference. Other methods for making human antibodies may also be produced by transforming B-cells with EBV and subsequent cloning of secretors as described by Hoon et al., (1993).

It is now possible to produce transgenic animals (e.g., mice) that are capable, upon immunization, of producing a repertoire of human antibodies in the absence of endogenous immunoglobulin production. For example, it has been described that the homozygous deletion of the antibody heavy chain joining region (J_{H}) gene in chimeric and germ-line mutant mice results in complete inhibition of endogenous antibody production. Transfer of the human germ-line immunoglobulin gene array in such germ-line mutant mice will result in the production of human antibodies upon antigen challenge (see, Jakobovits et al., 1993; Jakobovits et al., 1993).

Phage Display. Alternatively, the phage display technology (McCafferty et al., 1990) can be used to produce antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

Because the filamentous particle contains a single-stranded DNA copy of the phage genome, selections based on the functional properties of the antibody also result in selection of the gene encoding the antibody exhibiting those properties. Thus, the phage mimicks some of the properties of the B-cell. Phage display can be performed in a variety of formats; for their review see, Johnson et al., 1993. Several sources of V-gene segments can be used for phage display. Clackson et al., (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al. (1991), or Griffith et al. (1993).

In a natural immune response, antibody genes accumulate mutations at a high rate (somatic hypermutation). Some of the changes introduced will confer higher affinity, and B-cells displaying high-affinity surface immunoglobulin are preferentially replicated and differentiated during subsequent antigen challenge. This natural process can be mimicked by employing the technique known as “chain shuffling” (Marks et al., 1992). In this method, the affinity of “primary” human antibodies obtained by phage display can be improved by sequentially replacing the heavy and light chain V region genes with repertoires of naturally occurring variants (repertoires) of V domain genes obtained from unimmunized donors. This techniques allows the production of antibodies and antibody fragments with affinities in the nM range. A strategy for making very large phage antibody repertoires has been described by Waterhouse et al. (1993), and the isolation of a high affinity human antibody directly from such large phage library is reported by Griffith et al. (1994). Gene shuffling can also be used to derive human antibodies from rodent antibodies, where the human antibody has similar affinities and specificities to the starting rodent antibody. According to this method, which is also referred to as “epitope imprinting”, the heavy or light chain V domain gene of rodent antibodies obtained by phage display technique is replaced with a repertoire of human V domain genes, creating rodent-human chimeras. Selection on antigen results in isolation of human variable capable of restoring a functional antigen-binding site, i.e. the epitope governs (imprints) the choice of partner. When the process is repeated in order to replace the remaining rodent V domain, a human antibody is obtained (PCT patent application WO 93/06213). Unlike traditional humanization of rodent antibodies by CDR grafting, this technique provides completely human antibodies, which have no framework or CDR residues of rodent origin.

Antibody Conjugates. Antibody conjugates comprising an antibody of the invention linked to another agent, such as but not limited to a therapeutic agent, a detectable label, a cytotoxic agent, a chemical, a toxic, an enzyme inhibitor, a pharmaceutical agent, etc. form further aspects of the invention. Diagnostic antibody conjugates may be used both in in vitro diagnostics, as in a variety of immunoassays, and in in vivo diagnostics, such as in imaging technology.

Certain antibody conjugates include those intended primarily for use in vitro, where the antibody is linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Preferred secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

Other antibody conjugates, intended for functional utility, include those where the antibody is conjugated to an enzyme inhibitor such as an adenosine deaminase inhibitor, or a dipeptidyl peptidase IV inhibitor.

Radiolabeled Antibody Conjugates. In using an antibody-based molecule as an in vivo diagnostic agent to provide an image of, for example, brain, thyroid, breast, gastric, colon, pancreas, renal, ovarian, lung, prostate, hepatic, and lung cancer or respective metastases, magnetic resonance imaging, X-ray imaging, computerized emission tomography and such technologies may be employed. In the antibody-imaging constructs of the invention, the antibody portion used will generally bind to the cancer marker or other secreted and/or transmembrane protein and the imaging agent will be an agent detectable upon imaging, such as a paramagnetic, radioactive or fluorescent agent.

Many appropriate imaging agents are known in the art, as are methods for their attachment to antibodies (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). MAbs also may be reacted with an enzyme in the presence of a coupling agent such as glutaraldehyde or periodate. Conjugates with fluorescein markers are prepared in the presence of these coupling agents or by reaction with an isothiocyanate.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being preferred for use in certain embodiments, and technicium^{99m} and indium¹¹ are also often preferred due to their low energy and suitability for long range detection.

Radioactively labeled mAbs of the present invention may be produced according to well-known methods in the art. For instance, mAbs can be iodinated by contact with sodium or potassium iodide and a chemical oxidizing agent such as sodium hypochlorite, or an enzymatic oxidizing agent, such as lactoperoxidase. MAbs according to the invention may be labeled with technetium-^{99m} by ligand exchange process, for example, by reducing pertechnate with stannous solution, chelating the reduced technetium onto a Sephadex column and applying the antibody to this column or by direct labeling techniques, e.g., by incubating pertechnate, a reducing agent such as SNCl₂, a buffer solution such as sodium-potassium phthalate solution, and the antibody.

Intermediary functional groups which are often used to bind radioisotopes which exist as metallic ions to antibody are diethylenetriaminepentaacetic acid (DTPA) and ethylene diaminetetracetic acid (EDTA).

Fluorescent labels include rhodamine, fluorescein isothiocyanate and renographin.

K. Immunological Detection

Immunoassays. The antibodies of the invention are contemplated to be useful in various diagnostic and prognostic applications connected with the detection and analysis of cancer, obesity and a host of other diseases such as but not limited to heart disease, osteoporosis, diabetes, and neurodegenerative diseases. In still further embodiments, the present invention thus contemplates immunodetection methods for binding, purifying, identifying, removing, quantifying or otherwise generally detecting biological components.

The steps of various useful immunodetection methods have been described in the scientific literature, such as, e.g., Nakamura et al. 1987, incorporated herein by reference. Immunoassays, in their most simple and direct sense, are binding assays. Certain preferred immunoassays are the various types of enzyme linked immunosorbent assays (ELISAs), radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

In general, immunobinding methods include obtaining a sample suspected of containing a protein, peptide or antibody, and contacting the sample with an antibody or protein or peptide in accordance with the present invention, as the case may be, under conditions effective to allow the formation of immunocomplexes.

The immunobinding methods of this invention include methods for detecting or quantifying the amount of a reactive component in a sample, which methods require the detection or quantitation of any immune complexes formed during the binding process. Here, one would obtain a sample suspected of containing a disease marker antigen or cancer marker protein, peptide or a corresponding antibody, and contact the sample with an antibody or encoded protein or peptide, as the case may be, and then detect or quantify the amount of immune complexes formed under the specific conditions.

In terms of antigen detection, the biological sample analyzed may be any sample that is suspected of containing a cancer-specific antigen, such as a T-cell cancer, melanoma, glioblastoma, astrocytoma, a cancer of the breast, gastric, colon, pancreas, renal, ovarian, lung, prostate, hepatic, lung, lymph node or bone marrow tissue section or specimen, a homogenized tissue extract, an isolated cell, a cell membrane preparation, separated or purified forms of any of the above protein-containing compositions, or even any biological fluid that comes into contact with cancer tissues, including blood, lymphatic fluid, seminal fluid and urine.

Contacting the chosen biological sample with the protein, peptide or antibody under conditions effective and for a period of time sufficient to allow the formation of immune complexes (primary immune complexes) is generally a matter of simply adding the composition to the sample and incubating the mixture for a period of time long enough for the antibodies to form immune complexes with, i.e., to bind to any antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any radioactive, fluorescent, biological or enzymatic tags or labels of standard use in the art. References concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

The encoded protein, peptide or corresponding antibody employed in the detection may itself be linked to a detectable label, wherein one would then simply detect this label, thereby allowing the amount of the primary immune complexes in the composition to be determined.

Alternatively, the first added component that becomes bound within the primary immune complexes may be detected by means of a second binding ligand that has binding affinity for the encoded protein, peptide or corresponding antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under conditions effective and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the encoded protein, peptide or corresponding antibody is used to form secondary immune complexes, as described above. After washing, the secondary immune complexes are contacted with a third binding ligand or antibody that has binding affinity for the second antibody, again under conditions effective and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

The immunodetection methods of the present invention have evident utility in the diagnosis of cancer. Here, a biological or clinical sample that might contain either the encoded protein or peptide or corresponding antibody is used. However, these embodiments also have applications to non-clinical samples, such as in the titering of antigen or antibody samples, in the selection of hybridomas, and the like.

As noted, it is contemplated that an immunodetection technique such as an ELISA, immunohistochemistry, FACS scanning, in vivo imaging, may be useful in conjunction with detecting presence of a disease antigen, identified by the methods of the invention, on a clinical sample. The skilled artisan is well versed in these techniques.

L. Kits

Cancer Detection Kits. The materials and reagents required for detecting the levels of expression of a polypeptide/protein comprising a signal sequence and/or a transmembrane sequence identified by methods of the invention in a biological sample which is isolated from a subject with a disease or a particular physiological state or a condition etc., may be assembled together in a kit.

Molecular Biology Kits. One set of kits are designed to detect the levels of expression of a polypeptide/protein comprising a signal sequence and/or a transmembrane sequence expressed differentially in a cancer cell versus a normal cell. Thus, the kits are designed to detect cancer markers identified by the invention. Preferably, the kits will comprise, in suitable container, one or more nucleic acid probes or primers and means for detecting nucleic acids. Therefore, kits for diagnosing cancer will comprise, a) oligonucleotide probes comprising a sequence comprised within one of SEQ ID NO: 17, SEQ ID NO: 23, SEQ ID NO: 27, SEQ ID NO: 37, SEQ ID NO: 43, SEQ ID NO: 47, SEQ ID NO: 53, SEQ ID NO: 71, SEQ ID NO: 73, SEQ ID NO: 75, SEQ ID NO: 77, SEQ ID NO: 83, SEQ ID NO: 85, SEQ ID NO: 91, SEQ ID NO: 93, SEQ ID NO: 97, SEQ ID NO: 99, SEQ ID NO: 103, SEQ ID NO: 109, SEQ ID NO: 111, SEQ ID NO: 125, SEQ ID NO: 129, or SEQ ID NO: 3, SEQ ID NO: 7, SEQ ID NO: 9, SEQ ID NO: 13, SEQ ID NO: 15, SEQ ID NO: 19, SEQ ID NO: 25, SEQ ID NO: 29, SEQ ID NO: 31, SEQ ID NO: 33, SEQ ID NO: 35, SEQ ID NO: 39, SEQ ID NO: 41, SEQ ID NO: 49, SEQ ID NO: 51, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 79, SEQ ID NO: 81, SEQ ID NO: 87, SEQ ID NO: 95, SEQ ID NO: 101, SEQ ID NO: 113, SEQ ID NO: 115,SEQ ID NO: 119, SEQ ID NO: 121, SEQ ID NO: 127, or a complement thereof; and b) reagents, enzymes and buffers, enclosed in a suitable container means.

In certain embodiments, such as in kits for use in Northern blotting, the means for detecting the nucleic acids may be a label, such as a radiolabel, that is linked to a nucleic acid probe itself.

Preferred kits are those suitable for use in PCR. In PCR kits, two primers will preferably be provided that have sequences from, and that hybridize to, spatially distinct regions of the genes corresponding to a polypeptide/protein comprising a signal sequence and/or a transmembrane sequence expressed differentially in a cancer cell versus a normal cell to be identified. Preferred pairs of primers for amplifying nucleic acids are selected to amplify the sequences specified herein. Also included in PCR kits may be enzymes suitable for amplifying nucleic acids, including various polymerases (RT, Taq, etc.), deoxynucleotides and buffers to provide the necessary reaction mixture for amplification.

The molecular biological detection kits of the present invention, as disclosed herein, also may contain one or more of a variety of other cancer marker gene sequences as described above. By way of example only, one may mention prostate specific antigen (PSA) sequences, probes and primers.

In each case, the kits will preferably comprise distinct containers for each individual reagent and enzyme, as well as for each cancer probe or primer pair. Each biological agent will generally be suitable aliquoted in their respective containers.

The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

Immunodetection Kits. In further embodiments, the invention provides immunological kits for use in detecting the levels of expression of a polypeptide/protein comprising a signal sequence and/or a transmembrane sequence expressed differentially in a cancer cell versus a normal cell in biological samples. Such kits will generally comprise one or more antibodies that have immunospecificity for the polypeptide/protein comprising a signal sequence and/or a transmembrane sequence that is a cancer marker.

The kit generally comprises, a) a pharmaceutically acceptable carrier; b) an antibody directed against an antigen encoded by SEQ ID NO: 18, SEQ ID NO: 24, SEQ ID NO: 28, SEQ ID NO: 38, SEQ ID NO: 44, SEQ ID NO: 48, SEQ ID NO: 54, SEQ ID NO: 72, SEQ ID NO: 74, SEQ ID NO: 76, SEQ ID NO: 78, SEQ ID NO: 84, SEQ ID NO: 86, SEQ ID NO: 92, SEQ ID NO: 94, SEQ ID NO: 98, SEQ ID NO: 100, SEQ ID NO: 104, SEQ ID NO: 110, SEQ ID NO: 1121, SEQ ID NO: 126, SEQ ID NO: 130, or SEQ ID NO: 4, SEQ ID NO: 8, SEQ ID NO: 10, SEQ ID NO: 14, SEQ ID NO: 16, SEQ ID NO: 20, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 50, SEQ ID NO: 52, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 80, SEQ ID NO: 82, SEQ ID NO: 88, SEQ ID NO: 96, SEQ ID NO: 102, SEQ ID NO: 114, SEQ ID NO: 116, SEQ ID NO: 120, SEQ ID NO: 122, SEQ ID NO: 128, or a fragment thereof, in a suitable container means; and c) an immunodetection reagent. MAbs are readily prepared and will often be preferred. Where proteins or peptides are provided, it is generally preferred that they be highly purified.

In certain embodiments, the antigen or the antibody may be bound to a solid support, such as a column matrix or well of a microtitre plate. The immunodetection reagents of the kit may take any one of a variety of forms, including those detectable labels that are associated with, or linked to, the given antibody or antigen itself Detectable labels that are associated with or attached to a secondary binding ligand are also contemplated. Exemplary secondary ligands are those secondary antibodies that have binding affinity for the first antibody or antigen.

Further suitable immunodetection reagents for use in the present kits include the two-component reagent that comprises a secondary antibody that has binding affinity for the first antibody or antigen, along with a third antibody that has binding affinity for the second antibody, wherein the third antibody is linked to a detectable label.

As noted above in the discussion of antibody conjugates, a number of exemplary labels are known in the art and all such labels may be employed in connection with the present invention. Radiolabels, nuclear magnetic spin-resonance isotopes, fluorescent labels and enzyme tags capable of generating a colored product upon contact with an appropriate substrate are suitable examples.

The kits may contain antibody-label conjugates either in fully conjugated form, in the form of intermediates, or as separate moieties to be conjugated by the user of the kit.

The kits may further comprise a suitably aliquoted composition of an antigen whether labeled or unlabeled, as may be used to prepare a standard curve for a detection assay.

The kits of the invention, regardless of type, will generally comprise one or more containers into which the biological agents are placed and, preferably, suitable aliquoted. The components of the kits may be packaged either in aqueous media or in lyophilized form.

The immunodetection kits of the invention, may additionally contain one or more of a variety of other cancer marker antibodies or antigens, if so desired. Such kits could thus provide a panel of cancer markers, as may be better used in testing a variety of patients. By way of example, such additional markers could include, other tumor markers such as PSA, SeLe^{X}, HCG, as well as p53, cyclin D1, p16, tyrosinase, MAGE, BAGE, PAGE, MUC18, CEA, p27, βHCG or other markers as identified and provided by the present invention.

The container means of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present invention will also typically include a means for containing the antibody, antigen, and any other reagent containers in close confinement for commercial sale. Such containers may include injection or blow-molded plastic containers into which the desired vials are retained.

Kits for Diagnosing Fat Metabolism Related Disorders. The materials and reagents required for detecting the levels of expression of a polypeptide/protein comprising a signal sequence and/or a transmembrane sequence identified by methods of the invention in a biological sample which is isolated from a subject with a disease or a particular physiological state or a condition etc., such as a metabolic disorder associated with the metabolism of fat, may be assembled together in a kit.

Molecular Biology Kits. One set of kits are designed to detect the levels of expression of a polypeptide/protein comprising a signal sequence and/or a transmembrane sequence expressed differentially in a various fat cells. Thus, the kits are designed to detect fat cell metabolism identified by the invention. Preferably, the kits will comprise, in suitable container, one or more nucleic acid probes or primers and means for detecting nucleic acids. Therefore, the kits for diagnosing fat cell metabolism will comprise, a) oligonucleotide probes comprising a sequence comprised within one of SEQ ID NO: 131, SEQ ID NO: 134, SEQ ID NO: 138, SEQ ID NO: 139, SEQ ID NO: 141, SEQ ID NO: 143, SEQ ID NO: 144, SEQ ID NO: 146, SEQ ID NO: 148, SEQ ID NO: 149, SEQ ID NO: 151, SEQ ID NO: 156, SEQ ID NO: 158, SEQ ID NO: 160, SEQ ID NO: 162, SEQ ID NO: 164, SEQ ID NO: 167, SEQ ID NO: 169, SEQ ID NO: 171, SEQ ID NO: 173, SEQ ID NO: 175, SEQ ID NO: 181, SEQ ID NO: 187, SEQ ID NO: 189, SEQ ID NO: 191, SEQ ID NO: 196, SEQ ID NO: 198, SEQ ID NO: 200, SEQ ID NO: 208, SEQ ID NO: 209, SEQ ID NO: 213, SEQ ID NO: 217, SEQ ID NO: 233, SEQ ID NO: 237, SEQ ID NO: 239, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 256, SEQ ID NO: 257, SEQ ID NO: 265, SEQ ID NO: 267, SEQ ID NO: 269, SEQ ID NO: 275, SEQ ID NO: 277, SEQ ID NO: 279, SEQ ID NO: 285, SEQ ID NO: 287, SEQ ID NO: 294, SEQ ID NO: 296, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, or a complement thereof; and b) reagents, enzymes and buffers, enclosed in a suitable container means.

In certain embodiments, such as in kits for use in Northern blotting, the means for detecting the nucleic acids may be a label, such as a radiolabel, that is linked to a nucleic acid probe itself.

Preferred kits are those suitable for use in PCR. In PCR kits, two primers will preferably be provided that have sequences from, and that hybridize to, spatially distinct regions of the genes corresponding to a polypeptide/protein comprising a signal sequence and/or a transmembrane sequence expressed differentially in a fat cell with an abnormal physiology or metabolism versus a normal fat cell to be identified. Preferred pairs of primers for amplifying nucleic acids are selected to amplify the sequences specified herein. Also included in PCR kits may be enzymes suitable for amplifying nucleic acids, including various polymerases (RT, Taq, etc.), deoxynucleotides and buffers to provide the necessary reaction mixture for amplification.

In each case, the kits will preferably comprise distinct containers for each individual reagent and enzyme, as well as for each probe or primer pair. Each biological agent will generally be suitable aliquoted in their respective containers.

The container means of the kits will generally include at least one vial or test tube. Flasks, bottles and other container means into which the reagents are placed and aliquoted are also possible. The individual containers of the kit will preferably be maintained in close confinement for commercial sale. Suitable larger containers may include injection or blow-molded plastic containers into which the desired vials are retained. Instructions may be provided with the kit.

Immunodetection Kits. In further embodiments, the invention provides immunological kits for use in detecting the levels of expression of a polypeptide/protein comprising a signal sequence and/or a transmembrane sequence expressed differentially in a fat cell that has a fat metabolic defect or other abnormal condition versus a normal fat cell in biological samples. Such kits will generally comprise one or more antibodies that have immunospecificity for the polypeptide/protein comprising a signal sequence and/or a transmembrane sequence that is expressed by a fat cell with a metabolic defect or physiological condition.

The kit generally comprises, a) a pharmaceutically acceptable carrier; b) an antibody directed against an antigen encoded by SEQ ID NO: 132, SEQ ID NO: 135, SEQ ID NO: 140, SEQ ID NO: 142, SEQ ID NO: 145, SEQ ID NO: 147, SEQ ID NO: 150, SEQ ID NO: 157, SEQ ID NO: 159, SEQ ID NO: 161, SEQ ID NO: 163, SEQ ID NO: 165, SEQ ID NO: 166, SEQ ID NO: 168, SEQ ID NO: 170, SEQ ID NO: 172, SEQ ID NO: 174, SEQ ID NO: 176, SEQ ID NO: 182, SEQ ID NO: 188, SEQ ID NO: 190, SEQ ID NO: 192, SEQ ID NO: 197, SEQ ID NO: 199, SEQ ID NO: 201, SEQ ID NO: 210, SEQ ID NO: 214, SEQ ID NO: 218, SEQ ID NO: 234, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 266, SEQ ID NO: 268, SEQ ID NO: 270, SEQ ID NO: 276, SEQ ID NO: 278, SEQ ID NO: 280, SEQ ID NO: 286, SEQ ID NO: 288, SEQ ID NO: 295, SEQ ID NO: 297, or an antigenic fragment thereof, in a suitable container means; and c) an immunodetection reagent. MAbs are readily prepared and will often be preferred. Where proteins or peptides are provided, it is generally preferred that they be highly purified.

In certain embodiments, the antigen or the antibody may be bound to a solid support, such as a column matrix or well of a microtitre plate. The immunodetection reagents of the kit may take any one of a variety of forms, including those detectable labels that are associated with, or linked to, the given antibody or antigen itself Detectable labels that are associated with or attached to a secondary binding ligand are also contemplated. Exemplary secondary ligands are those secondary antibodies that have binding affinity for the first antibody or antigen.

Further suitable immunodetection reagents for use in the present kits include the two-component reagent that comprises a secondary antibody that has binding affinity for the first antibody or antigen, along with a third antibody that has binding affinity for the second antibody, wherein the third antibody is linked to a detectable label.

As noted above in the discussion of antibody conjugates, a number of exemplary labels are known in the art and all such labels may be employed in connection with the present invention. Radiolabels, nuclear magnetic spin-resonance isotopes, fluorescent labels and enzyme tags capable of generating a colored product upon contact with an appropriate substrate are suitable examples.

The kits may contain antibody-label conjugates either in fully conjugated form, in the form of intermediates, or as separate moieties to be conjugated by the user of the kit.

The kits may further comprise a suitably aliquoted composition of an antigen whether labeled or unlabeled, as may be used to prepare a standard curve for a detection assay.

The kits of the invention, regardless of type, will generally comprise one or more containers into which the biological agents are placed and, preferably, suitable aliquoted. The components of the kits may be packaged either in aqueous media or in lyophilized form.

The container of the kits will generally include at least one vial, test tube, flask, bottle, or even syringe or other container means, into which the antibody or antigen may be placed, and preferably, suitably aliquoted. Where a second or third binding ligand or additional component is provided, the kit will also generally contain a second, third or other additional container into which this ligand or component may be placed.

The kits of the present invention will also typically include a means for containing the antibody, antigen, and any other reagent containers in close confinement for commercial sale. Such containers may include injection or blow-molded plastic containers into which the desired vials are retained.

### M. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Construction of Vector

One of the vectors of the invention is a plasmid based vector, peCAST which is illustrated in FIG. 1. This vector was constructed using the plasmid pCRII-TOPO (Invitrogen, San Diego, Calif.). A sixty-nine nucleotide deletion at the extreme 5′-end of the ampicillin-resistance (Amp-R) was generated, which corresponds to 23 amino acids at the amino-terminal that begin at the starting methionine and comprise the native signal sequence that targets the Amp-R gene product to the extracellular space in the bacteria. A 20-base multiple cloning site was cloned in place of this 69-base deletion.

### Example 2

### Candidate Nucleic Acids

A random primed cDNA library is generated from the tissue or cell type of interest, and directionally cloned upstream of a marker that confers survival on selective media only in the presence of a mammalian signal sequence.

A vector was generated as described in Example 1 above and tested with the cDNA fragments that encoded both known secreted proteins and non-secreted proteins. On selection for the ampicillin resistance marker colony formation was observed only when the cDNA fragments encoded a protein comprising a signal sequence and/or a transmembrane domain.

### Example 3

### Secreted/Transmembrane Proteins from Breast Cancer

mRNA derived from mouse mammary tissue was prepared as the candidate nucleic acid and tested. One microgram of mRNA was sufficient to yield >40,000 putative signal-sequence containing cDNA clones. Ten clones were sequenced and all comprised signal sequences. Nine of these were identified as secreted proteins and one was identified to be a transmembrane proteins normally present in mammary tissue. The transmembrane protein identified, GlyCAM1, is a marker of breast differentiation (Dowbenko et al, 1993). This method was also performed with PCR amplified cDNA from small tissue samples, comparable in size to biopsy specimens, and again positive clones were identified.

Breast cancer cell lines and breast cancer cells were also analyzed for identification of proteins comprising signal sequences and/or transmembrane sequences and several such proteins have been identified (see SEQ ID NOS: 1-130 for the corresponding nucleic acid and amino acid seqeunces). Of these, SEQ ID NO: 17, SEQ ID NO: 23, SEQ ID NO: 27, SEQ ID NO: 37, SEQ ID NO: 43, SEQ ID NO: 47, SEQ ID NO: 53, SEQ ID NO: 71, SEQ ID NO: 73, SEQ ID NO: 75, SEQ ID NO: 77, SEQ ID NO: 83, SEQ ID NO: 85, SEQ ID NO: 91, SEQ ID NO: 93, SEQ ID NO: 97, SEQ ID NO: 99, SEQ ID NO: 103, SEQ ID NO: 109, SEQ ID NO: 111, SEQ ID NO: 125, SEQ ID NO: 129 are novel previously uncharacterized nucleic acid sequences. These correspond to the amnio acid sequences SEQ ID NO: 18, SEQ ID NO: 24, SEQ ID NO: 28, SEQ ID NO: 38, SEQ ID NO: 44, SEQ ID NO: 48, SEQ ID NO: 54, SEQ ID NO: 72, SEQ ID NO: 74, SEQ ID NO: 76, SEQ ID NO: 78, SEQ ID NO: 84, SEQ ID NO: 86, SEQ ID NO: 92, SEQ ID NO: 94, SEQ ID NO: 98, SEQ ID NO: 100, SEQ ID NO: 104, SEQ ID NO: 110, SEQ ID NO: 1121, SEQ ID NO: 126, SEQ ID NO: 130.

Additionally, the inventors contemplate analyzing thousands of positive clones from both breast cancer cell lines as well as from clinical samples of breast cancer cells. This requires a rapid method for DNA extraction. Therefore, the inventors have developed a high-throughtput 96-well mini-prep format that allows DNA to be isolated from greater than 1000 colonies per day. Similar experiments are contemplated for other cancers as well.

Differential expression of the secreted and/or cell-surface markers in cancerous cells versus normal tissue is an important consideration for the identification of cancer-markers. Hence, the signal sequence-containing clones from mouse tissue were analyzed for amenability to microarray analysis. For this analysis, DNA was obtained from the 96-well miniprep protocol and the plasmid insert was amplified in a high-throughput 96-well format PCR™. Following this DNA was spotted onto a microarray chip and the array was hybridized with two different probes. Differential expression of genes has been demonstrated. In one example, a probe from normal breast tissue (sample 1), produces a green color, while a probe from breast cancer tissue (sample 2), emits a red color. Hence, a clone that is expressed only in normal tissue emits a green signal while a clone expressed in the cancerous tissue emits a red signal. A yellow signal is generated if a clone is approximately equally expressed in both the normal and breast cancer samples.

It is also contemplated that the arrays will be hybridized with combinations of cDNA generated from various breast cancer cell lines, human breast cancers, and normal breast tissue to determine which molecules are consistently present at elevated or depressed levels in the breast cancers. This will be useful in developing the diagnostic embodiments of the invention. Additionally, cDNA from different stages of breast cancer will be used to probe the microarrays in order to identify molecules whose expression levels correlate with particular stages of breast cancer progression. This will be useful in developing the prognostic/diagnostic embodiments of the invention. All the clones may be sequenced.

It is contemplated that this technique may be employed to isolate signal sequence-containing proteins from any tissue or cell type or cancer-type or other disease type. The present inventors have used this technique to analyze breast cancer cells for the following reasons. First, breast cancers affect a significant percentage (˜10%) of the female population. Second, breast cancer frequently strikes at a young age; therefore, early detection is of paramount importance in increasing survival. Third, there are no generally useful blood screening tests for breast cancer. The present invention, identifies cancer surface marker proteins and/or cancer markers that are secreted into the blood stream and therefore provides these marker proteins to develop diagnostic/prognostic assays to diagnose breast cancers.

To verify that the candidate differentially expressed clones are expressed in human breast cancers, RT-PCR, Northern Blotting, and in situ hybridization analysis will be performed on sections of human breast cancers. Other tissues will also be analyzed for expression in order to determine specificity. It is also contemplated that antibodies will be generated against the proteins to provide a second level of screening to ensure that the proteins encoded by the differentially expressed clones are present within human breast cancers. Immunohistochemistry is another technique used by pathologists to evaluate human specimens and immunohistochemical methods are well known in the art.

### Example 4

### Identification of Other Signal/Transmembrane Proteins

This example concerns the development of methods for identifying secreted and cell-surface proteins expressed in breast cancers and other cancers. It is contemplated that random primed cDNA will be generated from breast cancer cell lines (such as MCF-7, SK-BR3, etc.) and from human breast cancer specimens as well.

Cell lines and human specimens each have experimental advantages. There are a variety of breast cancer cell lines available and from which large quantities of starting material can be obtained. In addition, identification of proteins that are expressed in breast cancer cell lines provides a well-established model system in which further experimentation can be conducted. However, there are inherent differences between cultured cells and three-dimensional cancers, presumably involving additional cell-cell and cell-environment interactions. Therefore, it is important to include breast cancer biopsies as a source of secreted and cell-surface molecules.

cDNA libraries generated from both sources will be ligated into the vector constructs of the invention in order to select for signal sequence and/or transmembrane sequence containing molecules. Two independent breast cancer cell line cDNA libraries have already been developed, each of which contains approximately 10,000 putative secreted and cell-surface molecules. cDNA libraries have been made for human breast cancer specimens. The positive clones identified by the methods of the invention will then be sequenced and subject to other identification and isolation methods.

### Example 54

### Signal/Transmembrane Proteins from Adipocytes

Numerous proteins comprising a signal sequence and/or a transmembrane sequence have also been identified from adipocytes. Adipocytes were chosen with the intention of identifying proteins involved in fat metabolism by the methods of the invention. Once identified these proteins are isolated and identified. Briefly this involves, isolating DNA is from a large number of positive clones (˜12,000), spotting the DNA onto a microarray, and identifying differential gene expression in biologically meaningful situations such as in fibroblasts versus adipocytes, lean mice versus obese mice, etc.

Methods. Libraries obtained from wild-type mouse fat, Ob/Ob mouse fat (i.e., leptin deficient), and from 3T3-LI cell lines were plated and induced to form adipocytes. The fibroblastic 3T3-LI cell line can be converted into fat cells under appropriate conditions. A high-throughput 96-well format miniprep was performed to extract DNA from approximately 3-10,000 clones from each of the three libraries. The clones were then sequenced for quality control and for gene discovery and identification.

For analysis of differential expression the clones were PCR amplified and spotted onto a microarray. The spotted clones were then probed with mRNA from 3T3-LI cells which are the uninduced fibroblasts and with probes from the induced adipocytes, as well as with probes from the different mouse fat models. All differentially expressed clones were sequenced.

Using the E. coli based screening system that utilizes the ampicillin resistance marker gene several fat metabolism-related genes. Briefly, a plasmid vector (peCAST) was generated in which the ampicillin-resistance gene's endogenous signal sequence was mutated and two restriction sites (EcoRI and BamHI) were replaced in this region. peCAST does not confer bacterial growth on ampicillin plates. A directional, random primed library from mouse fat was generated and cloned into peCAST and plated onto ampicillin. The resulting library contained ˜40,000 positives that survived on ampicillin. Minipreps were performed over 200 unique sequences were obtained with about 85% containing transmembrane and/or secreted proteins represented by the nucleic acid sequences including, SEQ ID NO: 134, SEQ ID NO: 138, SEQ ID NO: 139, SEQ ID NO: 141, SEQ ID NO: 143, SEQ ID NO: 144, SEQ ID NO: 151, SEQ ID NO: 156, SEQ ID NO: 158, SEQ ID NO: 160, SEQ ID NO: 162, SEQ ID NO: 171, SEQ ID NO: 173, SEQ ID NO: 175, SEQ ID NO: 181, SEQ ID NO: 187, SEQ ID NO: 189, SEQ ID NO: 198, SEQ ID NO: 200, SEQ ID NO: 208, SEQ ID NO: 209, SEQ ID NO: 213, SEQ ID NO: 217, SEQ ID NO: 233, SEQ ID NO: 241, SEQ ID NO: 243, SEQ ID NO: 245, SEQ ID NO: 247, SEQ ID NO: 249, SEQ ID NO: 251, SEQ ID NO: 253, SEQ ID NO: 257, SEQ ID NO: 265, SEQ ID NO: 267, SEQ ID NO: 269, SEQ ID NO: 277, SEQ ID NO: 279, SEQ ID NO: 285, SEQ ID NO: 287, SEQ ID NO: 296, SEQ ID NO: 300, SEQ ID NO: 301, SEQ ID NO: 302, SEQ ID NO: 303, SEQ ID NO: 304, SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO:316, SEQ ID NO: 317, SEQ ID NO:318, SEQ ID NO: 319, SEQ ID NO: 320, SEQ ID NO: 321, SEQ ID NO: 322, SEQ ID NO: 323, SEQ ID NO: 324, and the amino acid sequences, SEQ ID NO: 135, SEQ ID NO: 140, SEQ ID NO: 142, SEQ ID NO: 145, SEQ ID NO: 157, SEQ ID NO: 159, SEQ ID NO: 161, SEQ ID NO: 163, SEQ ID NO: 172, SEQ ID NO: 174, SEQ ID NO: 176, SEQ ID NO: 182, SEQ ID NO: 188, SEQ ID NO: 190, SEQ ID NO: 199, SEQ ID NO: 201, SEQ ID NO: 210, SEQ ID NO: 214, SEQ ID NO: 218, SEQ ID NO: 234, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 258, SEQ ID NO: 266, SEQ ID NO: 268, SEQ ID NO: 270, SEQ ID NO: 278, SEQ ID NO: 280, SEQ ID NO: 286, SEQ ID NO: 288, SEQ ID NO: 297. One clone is a member of the resistin family.

### Example 6

### Development of Immunological Diagnostic Tests

Another embodiment of the invention is the development of diagnostic tests utilizing the proteins comprising a signal sequence and/or a transmembrane sequence identified by the methods of the invention. Thus, radioimmunoassay (RIA) or enzyme-linked immunosorbent assay (ELISA) tests and the like will be developed to analyze serum from patients to determine whether any of the isolated clones could be potential candidates for a general blood-screening test. Although this example generally discusses the example of diagnostic/prognostic tests with respect to breast cancer, the methods of the example are also applicable to development of diagnostic/prognostic tests for other cancers, other diseases, physiological conditions, and/or metabolic states of a patient as well.

Antibodies that may be used to detect/diagnose/prognose breast or other cancers include those generated to the novel breast cancer signal sequence and/or transmembrane proteins identified by the screening methods of the present invention and in non-limiting examples these include antibodies directed against an antigen encoded by SEQ ID NO: 18, SEQ ID NO: 24, SEQ ID NO: 28, SEQ ID NO: 38, SEQ ID NO: 44, SEQ ID NO: 48, SEQ ID NO: 54, SEQ ID NO: 72, SEQ ID NO: 74, SEQ ID NO: 76, SEQ ID NO: 78, SEQ ID NO: 84, SEQ ID NO: 86, SEQ ID NO: 92, SEQ ID NO: 94, SEQ ID NO: 98, SEQ ID NO: 100, SEQ ID NO: 104, SEQ ID NO: 110, SEQ ID NO: 1121, SEQ ID NO: 126, SEQ ID NO: 130, or SEQ ID NO: 4, SEQ ID NO: 8, SEQ ID NO: 10, SEQ ID NO: 14, SEQ ID NO: 16, SEQ ID NO: 20, SEQ ID NO: 26, SEQ ID NO: 30, SEQ ID NO: 32, SEQ ID NO: 34, SEQ ID NO: 36, SEQ ID NO: 40, SEQ ID NO: 42, SEQ ID NO: 50, SEQ ID NO: 52, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 80, SEQ ID NO: 82, SEQ ID NO: 88, SEQ ID NO: 96, SEQ ID NO: 102, SEQ ID NO: 114, SEQ ID NO: 116, SEQ ID NO: 120, SEQ ID NO: 122, SEQ ID NO: 128, or a fragment thereof.

Antibodies that may be used to detect/diagnose/prognose metabolic conditions relating to adipocyte metabolism include those generated to the novel adipocyte signal sequence and/or transmembnrane proteins identified by the screening methods of the present invention and in non-limiting examples these include antibodies directed against an antigen encoded by SEQ ID NO: 132, SEQ ID NO: 135, SEQ ID NO: 140, SEQ ID NO: 142, SEQ ID NO: 145, SEQ ID NO: 147, SEQ ID NO: 150, SEQ ID NO: 157, SEQ ID NO: 159, SEQ ID NO: 161, SEQ ID NO: 163, SEQ ID NO: 165, SEQ ID NO: 166, SEQ ID NO: 168, SEQ ID NO: 170, SEQ ID NO: 172, SEQ ID NO: 174, SEQ ID NO: 176, SEQ ID NO: 182, SEQ ID NO: 188, SEQ ID NO: 190, SEQ ID NO: 192, SEQ ID NO: 197, SEQ ID NO: 199, SEQ ID NO: 201, SEQ ID NO: 210, SEQ ID NO: 214, SEQ ID NO: 218, SEQ ID NO: 234, SEQ ID NO: 238, SEQ ID NO: 240, SEQ ID NO: 242, SEQ ID NO: 244, SEQ ID NO: 246, SEQ ID NO: 248, SEQ ID NO: 250, SEQ ID NO: 252, SEQ ID NO: 254, SEQ ID NO: 256, SEQ ID NO: 258, SEQ ID NO: 266, SEQ ID NO: 268, SEQ ID NO: 270, SEQ ID NO: 276, SEQ ID NO: 278, SEQ ID NO: 280, SEQ ID NO: 286, SEQ ID NO: 288, SEQ ID NO: 295, SEQ ID NO: 297, or a fragment thereof.

Although the sections above describe breast cancer and adipocyte specific antibodies, one of skill in the art will recognize that one can generate antibodies to any transmembrane and/or signal sequence comprising protein identified by the methods of the invention and these antibodies may be used to diagnose/detect/prognose a variety of pathological/physiological/metabolic conditions.

ELISAs. As noted, it is contemplated that an immunodetection technique such as an ELISA may be useful in conjunction with detecting the presence of a cancer marker or a marker of any other disease state or physiological condition in a clinical sample.

Several ELISA formats are contemplated. In one exemplary ELISA, antibodies binding to the proteins identified by the invention are immobilized onto a selected surface exhibiting protein affinity, such as a well in a polystyrene microtiter plate. Then, a test composition (a clinical sample) that might contain the disease marker antigen, such as a blood sample, is added to the wells. After binding and washing to remove non-specifically bound immunocomplexes, the bound antigen may be detected.

Detection is generally achieved by the addition of a second antibody specific for the target protein, that is linked to a detectable label. This type of ELISA is a simple “sandwich ELISA”. Detection also may be achieved by the addition of a second antibody, followed by the addition of a third antibody that has binding affinity for the second antibody, with the third antibody being linked to a detectable label.

In another exemplary ELISA, the samples suspected of containing the disease marker antigen, are immobilized onto the well surface and then contacted with the antibodies of the invention. After binding and washing to remove non-specifically bound immune-complexes, the bound antibody is detected. Where the initial antibodies are linked to a detectable label, the immune-complexes may be detected directly. Again, the immune-complexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the disease marker antigen, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immune-complexes. These are described as follows:

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control human cancer and/or clinical or biological sample to be tested under conditions effective to allow immune-complex (antigen/antibody) formation. Detection of the immune-complex then requires a labeled secondary binding ligand or antibody, or a secondary binding ligand or antibody in conjunction with a labeled tertiary antibody or third binding ligand.

“Under conditions effective to allow immune-complex (antigen/antibody) formation” means that the conditions preferably include diluting the antigens and antibodies with solutions such as BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 h, at temperatures preferably on the order of 25° to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune-complexes between the test sample and the originally bound material, and subsequent washing, the occurrence of even minute amounts of immune-complexes may be determined.

To provide a detecting means, the second or third antibody will have an associated label to allow detection. This can be an enzyme that will generate color development upon incubating with an appropriate chromogenic substrate. Thus, for example, one will desire to contact and incubate the first or second immune-complex with a urease, glucose oxidase, alkaline phosphatase or hydrogen peroxidase-conjugated antibody for a period of time and under conditions that favor the development of further immune-complex formation (e.g., incubation for 2 h at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

In other embodiments, solution -phase competition ELISA is also contemplated. Solution phase ELISA involves attachment of a disease marker antigen, identified by methods of the present invention, to a bead, for example, a magnetic bead. The bead is then incubated with sera from human and animal origin. After a suitable incubation period to allow for specific interactions to occur, the beads are washed. The specific type of antibody is detected with an antibody indicator conjugate. The beads are washed and sorted. This complex is the read on an appropriate instrument (fluorescent, electroluminescent, spectrophotometer, depending on the conjugating moiety). The level of antibody binding can thus by quantitated and is directly related to the amount of signal present.

Immunohistochemistry. The antibodies against the disease marker antigens identified by methods of the present invention may be used in conjunction with both fresh-frozen and formalin-fixed, paraffin-embedded tissue blocks prepared for study by immunohistochemistry (IHC). The method of preparing tissue blocks from these particulate specimens has been successfully used in previous IHC studies of various prognostic factors, e.g., in breast, and is well known to those of skill in the art (Brown et al., 1990; Abbondanzo et al., 1990; Allred et al., 1990).

Permanent-sections may be prepared by a similar method involving rehydration of the 50 mg sample in a plastic microfuge tube; pelleting; resuspending in 10% formalin for 4 h fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

FACS Analyses. Fluorescent activated cell sorting, flow cytometry or flow microfluorometry provides the means of scanning individual cells for the presence of an disease marker antigen. The method employs instrumentation that is capable of activating, and detecting the excitation emissions of labeled cells in a liquid medium.

FACS is unique in its ability to provide a rapid, reliable, quantitative, and multiparameter analysis on either living or fixed cells. Cells would generally be obtained by biopsy, single cell suspension in blood or culture. FACS analyses may be useful when desiring to analyze a number of cancer antigens at a given time, e.g., to follow an antigen profile during disease progression.

In vivo Imaging. The invention also contemplates in vivo methods of imaging cancer using antibody conjugates. The term “in vivo imaging” refers to any non-invasive method that permits the detection of a labeled antibody, or fragment thereof, that specifically binds to cancer or other disease cells located in the body of an animal or human subject.

The imaging methods generally involve administering to an animal or subject an imaging-effective amount of a detectably-labeled disease/cancer-specific antibody or fragment thereof (in a pharmaceutically effective carrier), such as an anti-breast cancer marker antibody raised against a breast cancer marker antigen identified by the methods of the present invention, and then detecting the binding of the labeled antibody to the cancerous tissue. The detectable label is preferably a spin-labeled molecule or a radioactive isotope that is detectable by non-invasive methods.

An “imaging effective amount” is an amount of a detectably-labeled antibody, or fragment thereof, that when administered is sufficient to enable later detection of binding of the antibody or fragment to cancer tissue. The effective amount of the antibody-marker conjugate is allowed sufficient time to come into contact with reactive antigens that may be present within the tissues of the patient, and the patient is then exposed to a detection device to identify the detectable marker.

Antibody conjugates or constructs for imaging thus have the ability to provide an image of the tumor, for example, through magnetic resonance imaging, x-ray imaging, computerized emission tomography and the like. Elements particularly useful in Magnetic Resonance Imaging (“MRI”) include the nuclear magnetic spin-resonance isotopes ¹⁵⁷Gd, ⁵⁵Mn, ¹⁶²Dy, ⁵²Cr, and ⁵⁶Fe, with gadolinium often being preferred. Radioactive substances, such as technicium^{99m} or indium¹¹¹, that may be detected using a gamma scintillation camera or detector, also may be used. Further examples of metallic ions suitable for use in this invention are ¹²³I, ¹³¹I, ¹³¹I, ⁹⁷Ru, ⁶⁷Cu, ⁶⁷Ga, ¹²⁵I, ⁶⁸Ga, ⁷²As, ⁸⁹Zr, and ²⁰¹TI.

A factor to consider in selecting a radionuclide for in vivo diagnosis is that the half-life of a nuclide be long enough so that it is still detectable at the time of maximum uptake by the target, but short enough so that deleterious radiation upon the host, as well as background, is minimized. Ideally, a radionuclide used for in vivo imaging will lack a particulate emission, but produce a large number of photons in a 140-2000 keV range, which may be readily detected by conventional gamma cameras.

A radionuclide may be bound to an antibody either directly or indirectly by using an intermediary functional group. Intermediary functional groups which are often used to bind radioisotopes which exist as metallic ions to antibody are diethylenetriaminepentaacetic acid (DTPA) and ethylene diaminetetracetic acid (EDTA).

Administration of the labeled antibody may be local or systemic and accomplished intravenously, intra-arterially, via the spinal fluid or the like. Administration also may be intradermal or intracavitary, depending upon the body site under examination. After a sufficient time has lapsed for the labeled antibody or fragment to bind to the diseased tissue, in this case cancer tissue, for example 30 min to 48 h, the area of the subject under investigation is then examined by the imaging technique. MRI, SPECT, planar scintillation imaging and other emerging imaging techniques may all be used.

The distribution of the bound radioactive isotope and its increase or decrease with time is monitored and recorded. By comparing the results with data obtained from studies of clinically normal individuals, the presence and extent of the diseased tissue can be determined.

The exact imaging protocol will necessarily vary depending upon factors specific to the patient, and depending upon the body site under examination, method of administration, type of label used and the like. The determination of specific procedures is, however, routine to the skilled artisan. Although dosages for imaging embodiments are dependent upon the age and weight of patient, a one time dose of about 0.1 to about 20 mg, more preferably, about 1.0 to about 2.0 mg of antibody-conjugate per patient is contemplated to be useful.

### Example 7

### Screening Methods for Identifying Nucleic Acids Encoding Signal and/or Transmembrane Sequences

This example describes methods of screening candidate eukaryotic nucleic acids to identify nucleic acid sequences encoding a signal sequence and/or a transmembrane sequence. It is envisioned that this method will be useful in identifying novel signal sequence and/or a transmembrane sequence containing eukaryotic proteins which include secreted and cell-surface proteins. Generically, the method comprises the steps of a) contacting a bacterial cell with at least one plasmid comprising a candidate eukaryotic nucleic acid segment and a marker gene comprising a mutation in a region comprising a signal sequence and/or a transmembrane sequence of the marker gene; and b) screening for function or expression of the marker gene; where function or expression of the marker gene indicates that the candidate nucleic acid segment comprises a sequence that encodes a signal sequence and/or a transmembrane sequence.

Any marker gene that requires a signal sequence for its function or expression may be used. In one such embodiment, the bacterial cell used for the screening is an E. coli cell and the plasmid comprises an antibiotic resistance marker gene that requires a signal sequence for its function or expression. In one specific example, the antibiotic resistance marker gene is the ampicillin-resistance gene with a mutation in its endogenous signal sequence, for example, two restriction sites, such as EcoRI and BamHI, may replace 69 base pairs of the region comprising the endogenous signal sequence. This plasmid, embodied by peCAST, which is also described elsewhere in this specification, renders the bacterial cell harboring it devoid of ampicillin resistance.

As per the method of the invention, an eukaryotic nucleic acid molecule is then cloned into such a plasmid. For example, in the specific embodiment that utilizes peCAST as the plasmid, a eukaryotic nucleic acid molecule can be cloned into the EcoRI-BamHI site. If the eukaryotic nucleic acid molecule comprises a signal sequence and/or a transmembrane domain, it will restore a functional signal sequence in the plasmid marker gene. Thus, the function or expression of the marker gene will be restored. In the case of peCAST, the cloning of an eukaryotic nucleic acid molecule that comprises a signal sequence and/or a transmembrane domain, confers ampicillin resistance and allows bacterial growth on ampicillin plates.

Therefore, according to the method of the invention, candidate eukaryotic nucleic acid molecules are generated and cloned into peCAST or other similar plasmids and plated onto ampicillin plates or on other antibiotic plates or on other media specifically designed to detect the marker gene. The positives clones that survive on ampicillin or express any other marker gene are then selected. Minipreps are then performed to isolate the DNA from the clones and the DNA so isolated is then sequenced to identify the nucleic acid sequences comprising a transmembrane and/or signal sequence domain. This is followed by steps to isolate or identify the corresponding protein.

It is contemplated that one may use as a starting material for a candidate eukaryotic nucleic acid, any eukaryotic cell, tissue, organ, cell line, specimen, or biological sample, to generate a DNA library that has the candidate nucleic acid sequences that one wishes to screen. The cells, tissues, or samples can additionally be obtained from animals or cells in different physiological or metabolic or genetic conditions. For example, one library can be from a normal healthy human cell while another can be from a human afflicted with a disease such as a cancer, or a genetic disorder, or a metabolic, endocrinological, or other disease. The DNA libraries may be cDNA libraries, genomic DNA libraries, oligonucleotide libraries, etc.

The positive clones identified by the methods of the invention will then be sequenced and subject to other identification and isolation methods by methods well known in the art. In one embodiment, the method can be used to identify differential gene expression in normal versus diseased cells or normal cells versus cells in different metabolic conditions and involves, isolating DNA from a large number of positive clones (˜12,000), spotting the DNA onto a microarray, and identifying the genes differentially expressed. Once the nucleic acid sequences are identified the corresponding proteins are isolated and identified.

### Example 8

### Development of Diagnostic Methods

The present invention also provides diagnostic methods for assaying for the presence of a disease, metabolic condition or abnormal physiological condition in a human subject using the signal sequence and/or transmembrane comprising proteins or nucleic acids of the invention.

As proteins that comprise a transmembrane sequence and/or a signal sequence are typically proteins that are either secreted from a cell or reside on the surface of a cell, they are ideal targets for blood tests for the diagnosis of diseases. The discovery of novel secreted and transmembrane proteins, by the methods of the invention as described above, provides numerous targets/markers to diagnose a wide variety of diseases and abnormal metabolic or physiological conditions.

Such a diagnostic method will generally comprise, a) obtaining an antibody directed against a polypeptide that comprises a transmembrane sequence and/or a signal sequence that is identified to be a target protein or a marker protein in a disease or condition, b) obtaining a sample from a human subject suspected to have the disease or condition; c) admixing the antibody with the sample; and d) assaying the sample for antigen-antibody binding, wherein the antigen-antibody binding indicates the disease or condition in the subject.

One of ordinary skill in the art will recognize that any antibody may be used for such a diagnostic procedure and includes either a polyclonal antibody or a monoclonal antibody. Assaying methods are also well known in the art. For example, the assaying method may be an immunoprecipitation reaction, a radioimmunoassay, an ELISA, a Western blot, an immunofluorescence assay, etc.

It is also envisioned that such antibodies may be assembled together as a diagnostic kit. Kits for diagnosis are described elsewhere in the specification. Briefly, they comprise at least one antibody directed against an antigen encoding a protein comprising a signal sequence and/or a transmembrane domain in a pharmaceutically acceptable medium in a suitable container means. Additional reagents, buffers, enzymes and other agents that are required for the assaying or detection may be supplied in the kits as well.

Yet other diagnostic methods are contemplated which use molecular biology detection methods. These methods detect the nucleic acid (mRNA or DNA) expression of a nucleic acid that encodes a secreted and transmembrane proteins that has been identified to be expressed in an disease, and/or abnormal metabolic and/or physiological condition, by the methods of the invention as described above. Such a method comprises a) obtaining an oligonucleotide probe comprising a sequence encoding a secreted and/or transmembrane protein that has been identified to be expressed in an disease and/or abnormal metabolic and/or physiological condition; and b) employing the probe in a PCR or other detection protocol, wherein hybridization of said probe to a sequence indicates the presence of the disease or condition.

The components for the diagnosis of a disease using the method set forth above may also be assembled together in a diagnostic kit and such a kit will comprise at least one oligonucleotide probe comprising a sequence encoding a secreted and transmembrane proteins that has been identified to be expressed in an disease, and/or abnormal metabolic and/or physiological condition and reagents, enzymes and buffers required for the detection enclosed in a suitable container means.

Some of the diseases or conditions contemplated to be detected include endocrine diseases, renal diseases, cardiovascular diseases, rheumatologic diseases, hematological diseases, neurological diseases, oncological diseases, pulmonary diseases, gasterointestinal diseases and a vast variety of abnormal metabolic or physiological diseases. Specific examples include cancer, Alzheimer's disease, osteoporosis, coronary artery disease, congestive heart failure, stroke, diabetes, and the like. It will be appreciated by one of ordinary skill in the art, that the methods of the invention are capable of identifying eukaryotic proteins and/or nucleic acids encoding or comprising transmembrane and/or secreted domains in any cell type. Therefore, proteins and nucleic acids that are differentially expressed in any disease state or condition can be identified by the present methods and used as diagnostic markers in the diagnostic methods set for the above to identify any disease or condition. Thus, the present invention is not limited to any specific proteins/nucleic acids and/or diseases/conditions.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents, which are both chemically and physiologically related, may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 3,817,837

U.S. Pat. No. 3,850,752

U.S. Pat. No. 3,939,350

U.S. Pat. No. 3,996,345

U.S. Pat. No. 4,196,265

U.S. Pat. No. 4,275,149

U.S. Pat. No. 4,277,437

U.S. Pat. No. 4,366,241

U.S. Pat. No. 4,472,509

U.S. Pat. No. 4,683,195

U.S. Pat. No. 4,683,202

U.S. Pat. No. 4,800,159

U.S. Pat. No. 4,816,567

U.S. Pat. No. 4,867,973

U.S. Pat. No. 4,883,750

U.S. Pat. No. 5,021,236

U.S. Pat. No. 5,279,721

U.S. Pat. No. 5,536,637

U.S. Pat. No. 5,565,332

U.S. Pat. No. 5,925,565

U.S. Pat. No. 5,928,906

U.S. Pat. No. 5,935,819

U.S. Pat. No. 6,060,249

Abbondanzo, Ann Diagn Pathol, 3(5):318-27, 1999.

Allred et al., Breast Cancer Res. Treat., 16: 182(#149), 1990.

Antibodies: A Laboratory Manual, Cold Spring Harbor Laboratory, 1988

Brodeur et al., “Monoclonal Antibody Production Techniques and Applications”, 51-63, Marcel Dekker, Inc., New York, 1987.

Brown et al., Breast Cancer Res. Treat., 16: 192(#191), 1990.

Carbonelli et al., FEMS Microbiol Lett, 177(1):75-82, 1999.

Clackson et al., Nature, 352:624-628, 1991.

Cocea, “Duplication of a region in the multiple cloning site of a plasmid vector to enhance cloning-mediated addition of restriction sites to a DNA fragment,” Biotechniques, 23(5):814-816, 1997.

Dowbenko, Kikuta, Fennie, Gillett, Lasky, “Glycosylation-dependent cell adhesion molecule 1 (GlyCAM 1) mucin is expressed by lactating mammary gland epithelial cells and is present in milk. J. Clin. Invest., 92(2): 952-960, 1993.

EPA No. 0244042

EPA No. 320,308

EPA No. 329,822

Fodor et al., Nature, 364:555-556, 1993.

Freifelder, Physical Biochemistry Applications to Biochemistry and Molecular Biology, 2nd ed. Wm. Freeman and Co., New York, N.Y., 1982.

Frohman, In: PCR Protocols: A Guide To Methods And Applications, Academic Press, N.Y., 1990.

GB No. 2,202,328

Gefter et al., Somatic Cell Genet, 3(2):231-6, 1977.

Goding, 1986, In: Monoclonal Antibodies: Principles and Practice, 2d ed., Academic Press, Orlando, Fla., pp. 60-61, and 71-74, 1986.

Griffith et al., EMBO J., 12:725-734, 1993.

Hacia, et al., Nature Genet., 14:441-449, 1996.

Hoon et al., J. Urol., 150(6):2013-2018, 1993.

Innis et al., PCR Protocols, Academic Press, Inc., San Diego Calif., 1990.

Jakobovits et al., Proc. Natl. Acad. Sci. USA, 90:2551-255, 1993.

Kaiser and Botstein, Mol. Cell. Biol., 6:2382-2391, 1986.

Kaiser et al., Science, 235:312-317, 1987.

Klein et al., Proc. Natl. Acad. Sci., 93:7108-7113, 1996.

Kohler and Milstein, Eur J Immunol, 6(7):511-9, 1976.

Kohler and Milstein, Nature, 256(5517):495-7, 1975.

Kozbor, J. Immunol., 133:3001, 1984.

Kwoh et al., Proc. Nat. Acad. Sci. USA, 86: 1173, 1989.

Levenson et al., Hum Gene Ther, 9(8): 1233-6, 1998.

Macejak and Sarnow, Nature, 353:90-94, 1991.

Marks et al., Bio/Technol., 10:779-783, 1992.

Marks et al., J. Mol. Biol., 222:581-597, 1991.

McCafferty et al., Nature, 348:552-553, 1990.

Millstein and Cuello, Nature, 305:537-539, 1983.

Nakamura et al., In: Handbook of Experimental Immunology (4^{th} Ed.), Weir, Herzenberg, Blackwell, Herzenberg, (eds). Vol. 1, Chapter 27, Blackwell Scientific Publ., Oxford, 1987.

Ohara et al., Proc. Nat'l Acad. Sci. USA, 86: 5673-5677, 1989.

PCT App. No. PCT/US89/01025

PCT Application WO 88/10315

PCT Application WO 89/06700

PCT Application WO 90/07641

PCT Application WO 93/06213

PCT Application WO 93/08829

Pease et al., Proc. Natl. Acad. Sci. USA, 91:5022-5026, 1994.

Pelletier and Sonenberg, Nature, 334:320-325, 1988.

Sambrook, Fritsch, Maniatis, Molecular Cloning: A Laboratory Manual, 2nd Ed., Cold Spring Harbor Press, Cold Spring Harbor, N.Y., 1989.

Shoemaker et al., Nature Genetics 14:450-456, 1996.

Steppan C M, Bailey S T, Bhat S, Brown E J, Banerjee R R, Wright C M, Patel H R, Ahima R S, Lazar M A, The hormone resistin links obesity to diabetes, Nature, Jan 18;409(6818):307-12 2001.

Steppan C M, Brown E J, Wright C M, Bhat S, Banerjee R R, Dai C Y, Enders G H, Silberg D G, Wen X, Wu G D, Lazar M A, A family of tissue-specific resistin-like molecules, Proc Natl Acad Sci USA, January 16;98(2):502-6 2001.

Steppan C M, Crawford D T, Chidsey-Frink K L, Ke H, Swick A G, Leptin is a potent stimulator of bone growth in ob/ob mice, Regul Pept, August 25;92(1-3):73-8 2000.

Friedman J M, Halaas J L, Leptin and the regulation of body weight in mammals, Nature, October 22;395(6704):763-70, 1998.

Suresh et al., Methods in Enzymology, 121:210, 1986.

Traunecker et al., EMBO, 10:3655-3659, 1991.

von Heijne, J. Mol. Biol., 184:99-105, 1985.

Walker et al., “Strand dis placement amplification—an isothermal, in vitro DNA amplification technique,” Nucleic Acids Res. 20(7):1691-1696, 1992.

Waterhouse et al., Nucl. Acids Res., 21:2265-2266, 1993.

Wu et al., Genomics, 4:560, 1989

What is claimed is:

1. A method of screening candidate eukaryotic nucleic acid for one or more nucleic acid sequence encoding a signal sequence and/or a transmembrane sequence comprising:
a) providing a bacterial cell;
b) contacting the bacterial cell with at least one plasmid comprising a candidate eukaryotic nucleic acid segment and a marker gene comprising a mutation in a region comprising a signal sequence and/or a transmembrane sequence of the marker gene; and
c) screening for function of the marker gene;
wherein function of the marker gene indicates that the candidate nucleic acid segment comprises a sequence that encodes a signal sequence and/or a transmembrane sequence.

2. The method of claim 1, wherein the nucleic acid is invertebrate nucleic acid.

3. The method of claim 2, wherein the invertebrate nucleic acid is fly nucleic acid.

4. The method of claim 2, wherein the invertebrate nucleic acid is C. elegans nucleic acid.

5. The method of claim 1, wherein the nucleic acid is vertebrate nucleic acid.

6. The method of claim 5, wherein the vertebrate nucleic acid is amphibian nucleic acid.

7. The method of claim 6, wherein said amphibian nucleic acid is frog nucleic acid.

8. The method of claim 5, wherein the vertebrate nucleic acid is reptile nucleic acid.

9. The method of claim 5, wherein said vertebrate nucleic acid is avian nucleic acid.

10. The method of claim 5, wherein the vertebrate nucleic acid is mammalian nucleic acid.

11. The method of claim 10, wherein the mammalian nucleic acid is mouse nucleic acid.

12. The method of claim 10, wherein the mammalian nucleic acid is human nucleic acid.

13. The method of claim 1, wherein the nucleic acid is fat cell nucleic acid.

14. The method of claim 12, wherein the nucleic acid is cancer cell nucleic acid.

15. The method of claim 14, wherein the cancer cell is obtained from a tumor or metastasis.

16. The method of claim 14, wherein the cancer cell is from an immortal cancer cell line.

17. The method of claim 14, wherein the cancer cell nucleic acid is breast cancer nucleic acid, hematological cancer nucleic acid, thyroid cancer nucleic acid, melanoma nucleic acid, T-cell cancer nucleic acid, B-cell cancer nucleic acid, ovarian cancer nucleic acid, pancreatic cancer nucleic acid, prostate cancer nucleic acid, colon cancer nucleic acid, bladder cancer nucleic acid, lung cancer nucleic acid, liver cancer nucleic acid, stomach cancer nucleic acid, testicular cancer nucleic acid, uterine cancer nucleic acid, brain cancer nucleic acid, lymphatic cancer nucleic acid, skin cancer nucleic acid, bone cancer nucleic acid, kidney cancer nucleic acid, rectal cancer nucleic acid, sarcoma nucleic acid, pituitary cancer nucleic acid, lipoma nucleic acid, adrenalcarcinoma nucleic acid; or nerve cell cancer nucleic acid.

18. The method of claim 17, wherein the cancer cell nucleic acid is breast cancer nucleic acid.

19. The method of claim 18, wherein the breast cancer cell nucleic acid is breast cancer cell line nucleic acid.

20. The method of claim 19, wherein the breast cancer cell line is an immortalized breast cancer cell line.

21. The method of claim 19, wherein the breast cancer cell line nucleic acid is MCF7 nucleic acid, SKBR-3 nucleic acid, MDA-MB-231 nucleic acid, MCF6 nucleic acid, T47D nucleic acid, or MDA-MB-435 nucleic acid.

22. The method of claim 18, wherein the breast cancer cell nucleic acid is a breast cancer sample.

23. The method of claim 1, wherein the nucleic acid is cultured cell nucleic acid.

24. The method of claim 1, wherein the nucleic acid is plant nucleic acid.

25. The method of claim 24, wherein the nucleic acid is corn, wheat, tobacco, arabidopsis, soybean, rice, or canola nucleic acid.

26. The method of claim 1, wherein the marker gene is further defined as a selectable marker gene comprising a mutation in a region comprising a signal sequence and/or a transmembrane sequence of the marker gene, and screening for function of the marker gene is further defined as assaying for survival of the cell or its progeny cells on the selectable media.

27. The method of claim 26, wherein survival of the cell or its progeny on selectable media indicates that the candidate nucleic acid sequence encodes a polypeptide comprising a signal sequence and/or a transmembrane sequence.

28. The method of claim 1, further comprising isolating at least one nucleic acid segment comprising a nucleic acid sequence encoding a polypeptide comprising a signal sequence and/or a transmembrane sequence from the candidate nucleic acid.

29. The method of claim 28, further defined as comprising isolating a plurality of nucleic acid segments comprising sequences encoding a polypeptide comprising a signal sequence and/or a transmembrane sequence from the candidate nucleic acid.

30. The method of claim 28, further comprising identifying at least one isolated nucleic acid segment.

31. The method of claim 30, wherein identifying comprises sequencing the nucleic acid sequence.

32. The method of claim 30, wherein identifying comprises expressing the nucleic acid sequence and identifying any polypeptides expressed.

33. The method of claim 32, wherein said identifying the polypeptides expressed is by using antibodies.

34. The method of claim 33, wherein the antibodies are prepared by phage display.

35. The method of claim 30, wherein identifying further comprises a cell-based assay.

36. The method of claim 30, wherein identifying further comprises a biochemistry-based assay.

37. The method of claim 28, further comprising characterization of at least one isolated nucleic acid segment.

38. The method of claim 37, further defined as comprising characterization of a plurality of isolated nucleic acid segments.

39. The method of claim 37, wherein the characterization comprises microarray analysis.

40. The method of claim 37, wherein the characterization comprises Northern blot analysis.

41. The method of claim 37, wherein the characterization comprises RT-PCR analysis.

42. The method of claim 37, wherein the characterization comprises expression of a polypeptide encoded by at least one candidate nucleic acid segment.

43. The method of claim 42, further defined as comprising analysis of function of the polypeptide.

44. The method of claim 40 further defined as comprising determining of antigenicity of the polypeptide.

45. The method of claim 37, wherein the characterization comprises determining whether the nucleic acid sequence or any polypeptide it encodes is an indicator of a disease, state of physiological condition, or other condition.

46. The method of claim 45, wherein the characterization comprises determining whether the isolated nucleic acid sequence or any polypeptide it encodes is an indicator of a disease.

47. The method of claim 46, wherein the disease is an endocrine disease, a renal disease, a cardiovascular disease, a rheumatologic disease, a hematological disease, a neurological disease, oncological, pulmonary, or a gastrointestinal disease.

48. The method of claim 47, wherein the disease is cancer, Alzheimer's disease, osteoporosis, coronary artery disease, congestive heart failure, stroke, or diabetes.

49. The method of claim 48, wherein the disease is cancer.

50. The method of claim 45, wherein the characterization comprises determining whether the isolated nucleic acid segment or any polypeptide it encodes is an indicator of a physiological condition.

51. The method of claim 50, wherein the state of physiological condition is a state of fat metabolism.

52. The method of claim 45, wherein characterization is further defined as determining whether the nucleic acid sequence or any polypeptide it encodes is an indicator that a subject has a disease, state of physiological condition, or other condition.

53. The method of claim 45, wherein characterization is further defined as determining whether the nucleic acid sequence or any polypeptide it encodes is an indicator that a subject has a propensity for a disease, state of physiological condition, or other condition.

54. The method of claim 45, further comprising determining that the nucleic acid sequence or any polypeptide it encodes is an indicator of a disease, state of physiological condition, or other condition.

55. The method of claim 54, further comprising assaying a subject for the nucleic acid sequence or any polypeptide it encodes to determine whether the subject has or has a propensity for a disease, state of physiological condition, or other condition.

56. The method of claim 55, further comprising determining that the subject has or has a propensity for a disease, state of physiological condition, or other condition.

57. The method of claim 1, wherein the bacterial cell is a gram negative bacterial cell.

58. The method of claim 1, wherein the bacterial cell is an Acetobacter cell, an Acinetobacter cell, a Bacillus cell, a Brevibacterium cell, a Campylobacter cell, a Citrobacter cell, a Clostridium cell, a Corynebacterium cell, an Enterobacter cell, an E. coli cell, a Heliobacter cell, a Klebsiella cell, a Lactobacillus cell, a Leuconostoc cell, a Micrococcus cell, a Pseudomonas cell, a Staphylococcus cell, a Streptococcus cell, a Thiobacillus cell or a Vibrio cell.

59. The method of claim 58, wherein the bacterial cell is an E. coli cell.

60. The method of claim 58, wherein the bacterial cell is a B. subtilis cell.

61. The method of claim 5 8, wherein the bacterial cell is a B. thuringiensis cell.

62. The method of claim 58, wherein the bacterial cell is a B. stearothermophilus cell.

63. The method of claim 58, wherein the bacterial cell is a B. licheniformis cell.

64. The method of claim 1, where the marker gene is a screenable marker gene.

65. The method of claim 64, wherein the screenable marker gene is detectable by fluorescence methods, colorimetric methods, radioactive, or enzymatic methods.

66. The method of claim 64, wherein the marker gene is a fluorescent protein gene or a beta-galactosidase gene.

67. The method of claim 1, where the marker gene is a scorable marker gene.

68. The method of claim 67, wherein the scorable marker gene is detectable by fluorescence methods, colorimetric methods, radioactive, or enzymatic methods.

69. The method of claim 1, where the marker gene is a measurable marker gene.

70. The method of claim 69, wherein the measurable marker gene is detectable by fluorescence methods, colorimetric methods, radioactive, or enzymatic methods.

71. The method of claim 1, where the marker gene is a selectable marker gene.

72. The method of claim 71, wherein the marker gene is an antibiotic resistance gene, a multidrug resistance gene, an herbicide resistance gene, or a toxin resistance gene.

73. The method of claim 71, where the marker gene is an antibiotic resistance gene.

74. The method of claim 73, where the antibiotic resistance gene is a beta-lactamase gene.

75. The method of claim 73, where the antibiotic resistance gene is an ampicillin-resistance gene, a penicillin-resistance gene, a cephalosporin-resistance gene, an oxacephem-resistance gene, a carbapenem-resistance gene, or a monobactam-resistance gene.

76. The method of claim 75, where the beta-lactamase gene is an ampicillin-resistance gene.

77. The method of claim 76, wherein the screening process comprises growth selection on selective media.

78. The method of claim 1, wherein the mutation is a deletion in the signal sequence of said marker gene.

79. The method of claim 1, wherein the mutation is a deletion of the entire signal sequence of said marker gene.

80. The method of claim 1, wherein the mutation is an insertion in the signal sequence of said marker gene.

81. The method of claim 1, wherein the mutation is a frameshift mutation in the signal sequence of said marker gene

82. The method of claim 1, wherein the mutation is a truncation of the signal sequence of said marker gene.

83. The method of claim 1, wherein the bacterial cell comprises a second marker gene.

84. The method of claim 83, wherein the second marker gene is a kanamycin resistance gene.

85. The method of claim 1, wherein the candidate nucleic acid is DNA.

86. The method of claim 85, wherein the candidate DNA is comprised in a DNA library.

87. The method of claim 86, wherein the DNA library is a genomic DNA library.

88. The method of claim 86, wherein the DNA library is an oligonucleotide library.

89. The method of claim 86, wherein the DNA library is a cDNA library.

90. The method of claim 86, wherein at least two members of the library are screened.

91. The method of claim 86, wherein at least 10 members of the library are screened.

92. The method of claim 86, wherein at least 100 members of the library are screened.

93. The method of claim 86, wherein at least 1000 members of the library are screened.

94. The method of claim 86, wherein at least 10,000 members of the library are screened.

95. The method of claim 86, wherein the entire library is screened.

96. The method of claim 1, wherein a cloning site is operably positioned in relation to the marker gene.

97. The method of claim 96, wherein the multiple cloning site comprises at least two restriction sites.

98. The method of claim 96, wherein the multiple cloning site comprises at least ten restriction sites.

99. The method of claim 96, wherein the multiple cloning site comprises at least one hundred restriction sites.

100. The method of claim 1, wherein the candidate nucleic acid is cloned into said plasmid by TA cloning.

101. A method of screening candidate nucleic acid for one or more nucleic acid sequences encoding a polypeptide comprising a signal sequence and/or a transmembrane sequence comprising:
a) providing a bacterial cell;
b) contacting the bacterial cell with at least one plasmid comprising a candidate nucleic acid segment and a marker gene comprising a mutation in a region comprising a signal sequence and/or a transmembrane sequence of the marker gene; and
c) screening for function of the marker gene;
wherein function of the marker gene indicates that the candidate nucleic acid segment comprises a sequence that encodes a polypeptide comprising a signal sequence and/or a transmembrane sequence.

102. A method of screening candidate nucleic acid for one or more nucleic acid sequences encoding a polypeptide comprising a signal sequence and/or a transmembrane sequence comprising:
a) providing a bacterial cell;
b) contacting the bacterial cell with at least one construct comprising a candidate nucleic acid segment and a mutated selectable marker gene comprising a mutation in a region comprising a signal sequence and/or a transmembrane sequence of the marker gene; and
c) screening for survival of the cell on selectable media;
wherein survival of the cell or its progeny cells on the selectable media indicates that the candidate nucleic acid segment comprises a sequence encoding a polypeptide comprising a signal sequence and/or a transmembrane sequence.

103. A construct for screening for nucleic acid sequences encoding a polypeptide comprising a signal sequence and/or a transmembrane sequence comprising:
a) a replication system functional in a bacterial host cell;
b) at least a first marker gene; and
c) a candidate nucleic acid sequence;
wherein expression of the marker gene in a bacterial cell indicates that the candidate nucleic acid sequence encodes a polypeptide comprising signal sequence and/or a transmembrane sequence.

104. The construct of claim 103, wherein the first marker gene is a screenable marker gene.

105. The construct of claim 103, where the first marker gene is a scorable marker gene.

106. The construct of claim 103, where the first marker gene is a measurable marker gene.

107. The construct of claim 103, where the first marker gene is a selectable marker gene.

108. The construct of claim 107, where the first marker gene is an antibiotic resistance gene.

109. The construct of claim 108, where the antibiotic resistance gene is an ampicillin-resistance gene.

110. The construct of claim 103, wherein the marker gene is mutated.

111. The construct of claim 103, wherein the construct further comprises a multiple cloning site.

112. The construct of claim 103, wherein the bacterial cell is a gram negative bacterial cell.

113. The construct of claim 112, wherein the bacterial host cell is an E. coli cell.
